SEQUENCE LISTING (100 sequences)

SEQ ID NO: 1 - protein, 13 aa, Artificial Sequence
  specificity domain
LARCWGTAETNVS

SEQ ID NO: 2 - protein, 13 aa, Artificial Sequence
  specificity domain
LNETWAVQGSPYL

SEQ ID NO: 3 - protein, 13 aa, Artificial Sequence
  specificity domain
ICQVIQNEQPHAK

SEQ ID NO: 4 - protein, 13 aa, Artificial Sequence
  specificity domain
MLKLLNQKKGPSQ

SEQ ID NO: 5 - protein, 13 aa, Artificial Sequence
  specificity domain
TTDVRPISGSRPV

SEQ ID NO: 6 - protein, 13 aa, Artificial Sequence
  specificity domain
FSTHRLPGCEPPC

SEQ ID NO: 7 - protein, 13 aa, Artificial Sequence
  specificity domain
ITQIGETSCGFFK

SEQ ID NO: 8 - protein, 13 aa, Artificial Sequence
  specificity domain
LHQWLETRPERQE

SEQ ID NO: 9 - protein, 13 aa, Artificial Sequence
  specificity domain
LQNYIPAHSLTLS

SEQ ID NO: 10 - protein, 13 aa, Artificial Sequence
  specificity domain
LQNYIPAQSLTLS

SEQ ID NO: 11 - protein, 13 aa, Artificial Sequence
  specificity domain
LHQWLETRPDRQE

SEQ ID NO: 12 - protein, 13 aa, Artificial Sequence
  specificity domain
FYLNWQDIPFLVQ

SEQ ID NO: 13 - protein, 13 aa, Artificial Sequence
  specificity domain
ITRWWEDLERDFP

SEQ ID NO: 14 - protein, 13 aa, Artificial Sequence
  specificity domain
IGEAWAKDSGLVR

SEQ ID NO: 15 - protein, 13 aa, Artificial Sequence
  specificity domain
LHRNWAPGGGPFP

SEQ ID NO: 16 - protein, 13 aa, Artificial Sequence
  specificity domain
FYLNWQDIPILTQ

SEQ ID NO: 17 - protein, 13 aa, Artificial Sequence
  specificity domain
FYLNWKDSPFLVQ

SEQ ID NO: 18 - protein, 13 aa, Artificial Sequence
  specificity domain
LHQWLETRPNRQV

SEQ ID NO: 19 - protein, 13 aa, Artificial Sequence
  specificity domain
FYLNWQDIPILTQ

SEQ ID NO: 20 - protein, 13 aa, Artificial Sequence
  specificity domain
LCLSWEEAQSPAN

SEQ ID NO: 21 - protein, 13 aa, Artificial Sequence
  specificity domain
LCLLWEDTLTPNC

SEQ ID NO: 22 - protein, 13 aa, Artificial Sequence
  specificity domain
FYLNWQDMAVLAQ

SEQ ID NO: 23 - protein, 13 aa, Artificial Sequence
  specificity domain
LSVSWKDLDDTFP

SEQ ID NO: 24 - protein, 13 aa, Artificial Sequence
  specificity domain
IQQSWLDLQELFP

SEQ ID NO: 25 - protein, 13 aa, Artificial Sequence
  specificity domain
LVSLSCHLDAELR

SEQ ID NO: 26 - protein, 13 aa, Artificial Sequence
  specificity domain
LVSLSYHLDTKVR

SEQ ID NO: 27 - protein, 13 aa, Artificial Sequence
  specificity domain
LCLRWEEGQAPKG

SEQ ID NO: 28 - protein, 13 aa, Artificial Sequence
  specificity domain
LCLCSEEGRAPMR

SEQ ID NO: 29 - protein, 13 aa, Artificial Sequence
  specificity domain
FYFKWQDIPIFTQ

SEQ ID NO: 30 - protein, 13 aa, Artificial Sequence
  specificity domain
IRRCWGQPEGPYA

SEQ ID NO: 31 - protein, 13 aa, Artificial Sequence
  specificity domain
LLRSWEEHNTPLS

SEQ ID NO: 32 - protein, 13 aa, Artificial Sequence
  specificity domain
FYFNWQDIPILTQ

SEQ ID NO: 33 - protein, 13 aa, Artificial Sequence
  specificity domain
FYLNWQDMAVVAQ

SEQ ID NO: 34 - protein, 13 aa, Artificial Sequence
  specificity domain
VAALAHPRTLALE

SEQ ID NO: 35 - protein, 11 aa, Artificial Sequence
  specificity domain
LTSWQESEGQG

SEQ ID NO: 36 - protein, 11 aa, Artificial Sequence
  specificity domain
ILRCLKVMGSY

SEQ ID NO: 37 - protein, 11 aa, Artificial Sequence
  specificity domain
ISQVGKGGGSV

SEQ ID NO: 38 - protein, 11 aa, Artificial Sequence
  specificity domain
MAALLSSSSPK

SEQ ID NO: 39 - protein, 11 aa, Artificial Sequence
  specificity domain
LTAWQESDGQG

SEQ ID NO: 40 - protein, 11 aa, Artificial Sequence
  specificity domain
GLRLKKALHAC

SEQ ID NO: 41 - protein, 11 aa, Artificial Sequence
  specificity domain
VRGRYEARQRK

SEQ ID NO: 42 - protein, 11 aa, Artificial Sequence
  specificity domain
LAAWQHSDSQT

SEQ ID NO: 43 - protein, 11 aa, Artificial Sequence
  specificity domain
LQECLKPKKPV

SEQ ID NO: 44 - protein, 11 aa, Artificial Sequence
  specificity domain
LDRSFRAQVFS

SEQ ID NO: 45 - protein, 11 aa, Artificial Sequence
  specificity domain
IATSLKNNKWT

SEQ ID NO: 46 - protein, 11 aa, Artificial Sequence
  specificity domain
VKTRYDTRQRK

SEQ ID NO: 47 - protein, 11 aa, Artificial Sequence
  specificity domain
ANKICEKRTPS

SEQ ID NO: 48 - protein, 11 aa, Artificial Sequence
  specificity domain
LRAWFASEQMI

SEQ ID NO: 49 - protein, 11 aa, Artificial Sequence
  specificity domain
LAQLADGGRVR

SEQ ID NO: 50 - protein, 11 aa, Artificial Sequence
  specificity domain
LQRSFKAQVFS

SEQ ID NO: 51 - protein, 11 aa, Artificial Sequence
  specificity domain
FQSTVEKASLC

SEQ ID NO: 52 - protein, 11 aa, Artificial Sequence
  specificity domain
AQRAADAAGPG

SEQ ID NO: 53 - protein, 11 aa, Artificial Sequence
  specificity domain
ANRICEKSEPE

SEQ ID NO: 54 - protein, 11 aa, Artificial Sequence
  specificity domain
GLRLKRQARAC

SEQ ID NO: 55 - protein, 11 aa, Artificial Sequence
  specificity domain
IVTALRSGNKE

SEQ ID NO: 56 - protein, 11 aa, Artificial Sequence
  specificity domain
IITALRSGNKE

SEQ ID NO: 57 - protein, 11 aa, Artificial Sequence
  specificity domain
FLTAMRESGAH

SEQ ID NO: 58 - protein, 11 aa, Artificial Sequence
  specificity domain
AILHEKKGDKM

SEQ ID NO: 59 - protein, 11 aa, Artificial Sequence
  specificity domain
LQPCLQVPSPL

SEQ ID NO: 60 - protein, 11 aa, Artificial Sequence
  specificity domain
ANLYDKVGYKV

SEQ ID NO: 61 - protein, 11 aa, Artificial Sequence
  specificity domain
VDLLFVRGAGN

SEQ ID NO: 62 - protein, 10 aa, Artificial Sequence
  specificity domain
IDKWSDRHRN

SEQ ID NO: 63 - protein, 10 aa, Artificial Sequence
  specificity domain
ALQHFRTTPR

SEQ ID NO: 64 - protein, 10 aa, Artificial Sequence
  specificity domain
ITAWCSSKAE

SEQ ID NO: 65 - protein, 10 aa, Artificial Sequence
  specificity domain
INEWMKRKIE

SEQ ID NO: 66 - protein, 10 aa, Artificial Sequence
  specificity domain
VKGASWLGKR

SEQ ID NO: 67 - protein, 10 aa, Artificial Sequence
  specificity domain
INQHLMNNKD

SEQ ID NO: 68 - protein, 10 aa, Artificial Sequence
  specificity domain
LERCLDHNAK

SEQ ID NO: 69 - protein, 10 aa, Artificial Sequence
  specificity domain
ALEHFRATPR

SEQ ID NO: 70 - protein, 10 aa, Artificial Sequence
  specificity domain
IHQSLEDNNR

SEQ ID NO: 71 - protein, 10 aa, Artificial Sequence
  specificity domain
MTLWFNREKT

SEQ ID NO: 72 - protein, 10 aa, Artificial Sequence
  specificity domain
IVRYLETNKY

SEQ ID NO: 73 - protein, 10 aa, Artificial Sequence
  specificity domain
IVRYLETSKY

SEQ ID NO: 74 - protein, 10 aa, Artificial Sequence
  specificity domain
LVKYLEENNT

SEQ ID NO: 75 - protein, 10 aa, Artificial Sequence
  specificity domain
VQEWSKNKAE

SEQ ID NO: 76 - protein, 10 aa, Artificial Sequence
  specificity domain
YSGWMERSSL

SEQ ID NO: 77 - protein, 10 aa, Artificial Sequence
  specificity domain
IRKFLSYKTQ

SEQ ID NO: 78 - protein, 10 aa, Artificial Sequence
  specificity domain
IVKYLQTSKY

SEQ ID NO: 79 - protein, 10 aa, Artificial Sequence
  specificity domain
IVRHFYYSNR

SEQ ID NO: 80 - protein, 10 aa, Artificial Sequence
  specificity domain
INKAMSYKPI

SEQ ID NO: 81 - protein, 10 aa, Artificial Sequence
  specificity domain
ITRALQVKKA

SEQ ID NO: 82 - protein, 10 aa, Artificial Sequence
  specificity domain
RIQESNGTWR

SEQ ID NO: 83 - protein, 10 aa, Artificial Sequence
  specificity domain
LRDSLKNANT

SEQ ID NO: 84 - protein, 10 aa, Artificial Sequence
  specificity domain
LRDSLKNANT

SEQ ID NO: 85 - DNA, 39 bp, Artificial Sequence
  primer
cccgctcgagtactagtatggatgaatcagccttgttgg

SEQ ID NO: 86 - DNA, 37 bp, Artificial Sequence
  primer
ggcgcggcggccgcttaggcacatggtaactgaggta

SEQ ID NO: 87 - DNA, 30 bp, Artificial Sequence
  primer
attacccgggatggcaaactcaatgaatgg

SEQ ID NO: 88 - DNA, 28 bp, Artificial Sequence
  primer
gggctcgagtcttctctgggcaagggga

SEQ ID NO: 89 - DNA, 56 bp, Artificial Sequence
  primer
cttacggcatggcaggagtcggatggtcagggctgtcccgagtgcaggactcttgt

SEQ ID NO: 90 - DNA, 56 bp, Artificial Sequence
  primer
gccctgaccatccgactcctgccatgccgtaagacatcgcttgcaaaacgtatgct

SEQ ID NO: 91 - DNA, 55 bp, Artificial Sequence
  primer
ttgctggggatcgtaggttctcgaaatgagctcagatgccctttctgtcgttgtg

SEQ ID NO: 92 - DNA, 54 bp, Artificial Sequence
  primer
tctgagctcatttcgagaacctacgatccccagcaagcaagaggtgcacatcaa

SEQ ID NO: 93 - DNA, 1140 bp, Artificial Sequence
  GST-hPOSH (codons 1-139)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccaggaattcccgggtcgagtactagtatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgcctaa

SEQ ID NO: 94 - protein, 379 aa, Artificial Sequence
  GST-hPOSH (codons 1-139)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGIPGSSTSMDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCA

SEQ ID NO: 95 - DNA, 2202 bp, Artificial Sequence
  GST-Cbl-b (codons 1-490)
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccaggaattcccgggatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggatccctttgatccaagagatgaaggctccaggtgttgcagcatcattgacccctttggcatgccgatgctcgacttggacgacgatgatgatcgtgaggagtccttgatgatgaatcggttggcaaacgtccgaaagtgcactgacaggcagaactcaccagtcacatcaccaggatcctctccccttgcccagagaagactcgagcggccgcatcgtgactga

SEQ ID NO: 96 - protein, 733 aa, Artificial Sequence
  GST-Cbl-b (codons 1-490)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGIPGMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRLERPHRD

SEQ ID NO: 97 - DNA, 1137 bp, Artificial Sequence
  GST-hPOSH chimeric RING domain
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccaggaattcccgggtcgagtactagtatggatgaatcagccttgttggatcttttggagtgtccggtgtgtctagagcgccttgatgcttctgcgaaggtcttgccttgccagcatacgttttgcaagcgatgtcttacggcatggcaggagtcggatggtcagggctgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgcctaa

SEQ ID NO: 98 - protein, 378 aa, Artificial Sequence
  GST-hPOSH chimeric RING domain
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGIPGSSTSMDESALLDLLECPVCLERLDASAKVLPCQHTFCKRCLTAWQESDGQGCPECRTLVGSGVEELPSNILLVRLLDGIKQRPWKPGPGGGSGTNCTNALRSQSSTVANCSSKDLQSSQGGQQPRVQSWSPPVRGIPQLPCA

SEQ ID NO: 99 - DNA, 2205 bp, Artificial Sequence
  GST-Cbl-b chimeric RING domain
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccaggaattcccgggatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgcttgctggggatcgtaggttctcgaaatgagctcagatgccctttctgtcgttgtgaaataaaaggaactgagcccataatcgtggatccctttgatccaagagatgaaggctccaggtgttgcagcatcattgacccctttggcatgccgatgctcgacttggacgacgatgatgatcgtgaggagtccttgatgatgaatcggttggcaaacgtccgaaagtgcactgacaggcagaactcaccagtcacatcaccaggatcctctccccttgcccagagaagactcgagcggccgcatcgtgactga

SEQ ID NO: 100 - protein, 734 aa, Artificial Sequence
  GST-Cbl-b chimeric RING domain
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGIPGMANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLLGIVGSRNELRCPFCRCEIKGTEPIIVDPFDPRDEGSRCCSIIDPFGMPMLDLDDDDDREESLMMNRLANVRKCTDRQNSPVTSPGSSPLAQRRLERPHRD

(19) United States
(12) Patent Application Publication
(10) Pub. No.: US 2006/0233779 A1
(43) Pub. Date: Oct. 19, 2006

(54) Ring finger family proteins and uses related thereto

(75) Inventors: Danny Ben-Avraham, Zichron Jackov (IL); Tsvika Greener, Ness-Ziona (IL); Liora Yaar, Raanana (IL)

Correspondence Address: FISH & NEAVE IP GROUP;ROPES & GRAY LLP, ONE INTERNATIONAL PLACE, BOSTON, MA, 02110-2624, US

(73) Assignee: Proteologics, Inc., Orangeburg, NY (US)

(21) Appl. No.: 11/320,326

(22) Filed: Dec. 27, 2005

Related U.S. Application Data
(63) Continuation of application No. PCT/US04/20988, filed on Jun. 28, 2004.
(60) Provisional application No. 60/483,133, filed on Jun. 27, 2003.

(51) Int. Cl. A61K 38/54; C12N 9/22; G01N 33/53

(52) U.S. Cl. 424/94.2; 435/199; 435/7.1

(57) ABSTRACT

The application provides, among other things, specificity domains, and methods for identifying and using specificity domains.

### CROSS-REFERENCE TO RELATED APPLICATIONS

This application is a continuation of International Application No. PCT/US2004/020988, entitled “Ring Finger Family Proteins and Uses Related Thereto”, filed Jun. 28, 2004, which claims the benefit of the filing date of U.S. Provisional Application No. 60/483,133, entitled “Ring Finger Family Proteins and Uses Related Thereto”, filed Jun. 27, 2003. The above-mentioned patent applications are incorporated herein by reference in their entirety.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes is a vital part of the drug design process. Many diseases and disorders could be treated or prevented by decreasing the expression of one or more genes involved in the molecular etiology of the condition if the appropriate molecular target could be identified and appropriate antagonists developed. For example, cancer is generally related to the unchecked progression of cell cycle processes and could be treated by agonizing or antagonizing appropriate cell cycle control genes. Furthermore many human genetic diseases, such as Huntington's disease, and certain prion conditions, which are influenced by both genetic and epigenetic factors, result from the inappropriate activity of a polypeptide as opposed to the complete loss of its function. Accordingly, antagonizing the aberrant function of such mutant genes would provide a means of treatment. Additionally, infectious diseases such as HIV have been successfully treated with molecular antagonists targeted to specific essential retroviral proteins such as HIV protease or reverse transcriptase. Drug therapy strategies for treating such diseases and disorders have frequently employed molecular antagonists which target the polypeptide product of the disease gene(s). However the discovery of relevant gene or protein targets is often difficult and time consuming.

It is well known in the art that ubiquitin-mediated proteolysis is the major pathway for the selective, controlled degradation of intracellular proteins in eukaryotic cells. Ubiquitin modification of a variety of protein targets within the cell appears to be important in a number of basic cellular functions such as regulation of gene expression, regulation of the cell-cycle, modification of cell surface receptors, biogenesis of ribosomes, and DNA repair. One major function of the ubiquitin-mediated system is to control the half-lives of cellular proteins. The half-life of different proteins can range from a few minutes to several days, and can vary considerably depending on the cell-type, nutritional and environmental conditions, as well as the stage of the cell-cycle.

Targeted proteins undergoing selective degradation, presumably through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1) and a ubiquitin-conjugating enzyme (E2), and in some instances may also require auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. In an initial ATP requiring step, a thioester is formed between the C-terminus of ubiquitin and an internal cysteine residue of an E1 enzyme. Activated ubiquitin is then transferred to a specific cysteine on one of several E2 enzymes. Finally, these E2 enzymes donate ubiquitin to protein substrates. Substrates are recognized either directly by ubiquitin-conjugated enzymes or by associated substrate recognition proteins, the E3 proteins, also known as ubiquitin ligases.

Accordingly, methods for identifying agents that affect the ubiquitin transfer pathway would be useful for, among other things, drug discovery programs.

### SUMMARY

Described herein are compositions and methods relating to E3 specificity domains. In certain embodiments, the invention relates to a RING finger E3 ligase selective inhibitor, wherein the RING finger E3 ligase selective inhibitor interacts with a specificity domain of a RING finger E3 ligase.

In additional embodiments, the invention relates to a RING finger E3 ligase selective inhibitor identified by a method comprising 3D (three dimensional) structure analysis of the interaction of a binding partner of a RING finger E3 ligase (e.g., a polypeptide such as a RING finger E3 ligase associated protein) with loop3 of the RING finger E3 ligase. In certain embodiments, the 3D structure is determined by homology modeling. In certain embodiments, the binding partner is an E2.

In additional embodiments, the invention provides a method of identifying the specificity domain of a RING Finger E3 ligase comprising identifying the RING domain of the RING finger E3 ligase and determining the position of the conserved Cysteine residues, wherein the specificity domain is the domain that lies between the 6^{th} and 7^{th} conserved Cysteine residue.

In yet other embodiments, the invention relates to an isolated or recombinant peptide consisting of a specificity domain. Examples of specificity domains include:

| | |
|---|---|
| LARCWGTAETNVS; | (SEQ ID NO:1) |
| LNETWAVQGSPYL; | (SEQ ID NO:2) |
| ICQVIQNEQPHAK; | (SEQ ID NO:3) |
| MLKLLNQKKGPSQ; | (SEQ ID NO:4) |
| TTDVRPISGSRPV; | (SEQ ID NO:5) |
| FSTHRLPGCEPPC; | (SEQ ID NO:6) |
| ITQIGETSCGFFK; | (SEQ ID NO:7) |
| LHQWLETRPERQE; | (SEQ ID NO:8) |
| LQNYIPAHSLTLS; | (SEQ ID NO:9) |
| LQNYIPAQSLTLS; | (SEQ ID NO:10) |
| LHQWLETRPDRQE; | (SEQ ID NO:11) |
| FYLNWQDIPFLVQ; | (SEQ ID NO:12) |
| ITRWWEDLERDFP; | (SEQ ID NO:13) |
| LTSWQESEGQG; | (SEQ ID NO:35) |
| ILRCLKVMGSY; | (SEQ ID NO:36) |
| ISQVGKGGGSV; | (SEQ ID NO:37) |
| MAALLSSSSPK; | (SEQ ID NO:38) |
| LTAWQESDGQG; | (SEQ ID NO:39) |
| GLRLKKALHAC; | (SEQ ID NO:40) |
| VRGRYEARQRK; | (SEQ ID NO:41) |
| LAAWQHSDSQT; | (SEQ ID NO:42) |
| LQECLKPKKPV; | (SEQ ID NO:43) |
| LDRSFRAQVFS; | (SEQ ID NO:44) |
| IATSLKNNKWT; | (SEQ ID NO:45) |
| VKTRYDTRQRK; | (SEQ ID NO:46) |
| ANKICEKRTPS; | (SEQ ID NO:47) |
| IDKWSDRHRN; | (SEQ ID NO:62) |
| ALQHFRTTPR; | (SEQ ID NO:63) |
| ITAWCSSKAE; | (SEQ ID NO:64) |
| INEWMKRKTE; | (SEQ ID NO:65) |
| VKGASWLGKR; | (SEQ ID NO:66) |
| INQHLMNNKD; | (SEQ ID NO:67) |
| LERCLDHNAK; | (SEQ ID NO:68) |
| ALEHFRATPR; | (SEQ ID NO:69) |
| IHQSLEDNNR; | (SEQ ID NO:70) |
| MTLWFNREKT; | (SEQ ID NO:71) |
| IVRYLETNKY; | (SEQ ID NO:72) |
| IVRYLETSKY; | (SEQ ID NO:73) |
| and | |
| LVKYLEENNT. | (SEQ ID NO:74) |

In additional embodiments, the invention relates to a fusion protein comprising a specificity domain and a second domain.

In certain embodiments, a specificity domain of the invention is hydrophobically modified.

The peptides of the invention may be modified at the N-terminal amino acid, C-terminal amino acid, or an internal amino acid may be modified. In certain embodiments, a peptide of the invention is modified at both the N-terminal amino acid and C-terminal amino acid.

In certain embodiments, a peptide of of the invention is modified with a fatty acid moiety that is selected from saturated and unsaturated fatty acids having between 2 and 24 carbon atoms.

In additional embodiments, a peptide of the invention is modified with a fatty acid moiety that is a myristoyl moiety. In other embodiments, a peptide of the invention is modified with a fatty acid moiety that is a palmitoyl moiety.

In additional embodiments, the invention relates to an antibody that interacts with a specificity domain of the invention.

In certain embodiments, the invention relates to a small molecule that interacts with a specificity domain of the invention. In yet other embodiments, the invention relates to a peptidomimetic that interacts with a specificity domain of the invention.

In additional embodiments, the invention relates to a method of inhibiting interaction of a RING Finger E3 ligase and a RING Finger E3 ligase associated protein, comprising administering an agent that interacts with the specificity domain of the RING finger E3 ligase. In certain embodiments, the agent is selected from the group consisting of: a small molecule, an antibody, and a peptidomimetic. In other embodiments, the agent selectively inhibits the ubiquitin ligase activity of the RING finger E3 ligase. In certain embodiments, the RING finger E3 ligase associated protein is selected from the group consisting of: an E2, an E3 ligase substrate, and a ubiquitin. In other embodiments, the agent selectively inhibits the interaction of the RING finger E3 ligase with one E2 over another E2. In additional embodiments, the agent selectively inhibits the ubiquitin ligase activity of the RING finger E3 ligase over another RING finger E3 ligase.

In yet other embodiments, the invention relates to a method of inhibiting the ubiquitin ligase activity of a RING finger E3 ligase, comprising administering an agent that interacts with the specificity domain of the RING finger E3 ligase. In certain embodiments, the agent selectively inhibits the ubiquitin ligase activity of the RING finger E3 ligase. In additional embodiments, the agent inhibits the interaction between the RING finger E3 ligase and a RING finger E3 ligase associated protein. In yet other embodiments, the RING finger E3 ligase associated protein is selected from the group consisting of: an E2, an E3 ligase substrate, and a ubiquitin. In further embodiments, the agent selectively inhibits the interaction of the RING finger E3 ligase with one E2 over another E2. In additional embodiments, the ability of the agent to selectively inhibit the ubiquitin ligase activity of the RING finger E3 ligase is at least 5 times greater than its ability to inhibit the ubiquitin ligase activity of another RING finger E3 ligase.

In additional embodiments, the invention relates to a method of screening for an agent that potentiates or inhibits the interaction between a RING finger E3 ligase and a RING finger E3 ligase associated protein, comprising: (a) providing a polypeptide comprising a portion of at least 20 amino acids of a RING finger E3 ligase, wherein the polypeptide includes at least a specificity domain of the RING finger E3 ligase; (b) providing a polypeptide comprising a portion of at least 20 amino acids of a RING finger E3 ligase associated protein; (c) providing a test agent; and (d) assaying for potentiation or inhibition of an interaction between the polypeptides of (a) and (b), wherein if the test agent inhibits or potentiates the interaction in (d), a test agent is identified that inhibits or potentiates the interaction between a RING finger E3 ligase and a RING finger E3 ligase associated protein. In certain embodiments, the RING finger E3 ligase associated protein is selected from the group consisting of: an E2, a RING finger E3 ligase substrate, and a ubiquitin. In additional embodiments, the agent selectively inhibits the interaction of the RING finger E3 ligase with one E2 over another E2.

In additional embodiments, the invention relates to a method of screening for an agent that inhibits the ubiquitin ligase activity of a RING finger E3 ligase, comprising: (a) providing a polypeptide comprising a portion of at least 20 amino acids of a RING finger E3 ligase, wherein the polypeptide includes at least a portion of the specificity domain of the RING finger E3 ligase; (b) providing an E1, an E2, and a ubiquitin; (c) providing a test agent; and (d) assaying for binding of the agent in (c) to at least a portion of the at least 20 amino acids of the polypeptide of (a).

In yet other embodiments, the invention provides a method of identifying an inhibitor of a RING finger E3 ligase, comprising: (a) identifying the specificity domain of the RING finger E3 ligase; and (b) identifying an agent that binds to at least a portion of the specificity domain identified in (a), wherein the agent identified in (b) is an inhibitor of the RING finger E3 ligase.

In certain embodiments, the inhibitor of the RING finger E3 ligase is a selective inhibitor.

In additional embodiments, the RING finger E3 ligase inhibitor is an inhibitor for therapeutic use. In further embodiments, the RING finger E3 ligase inhibitor is selected from the group consisting of: a small molecule, an antibody, and a peptidomimetic. In certain embodiments, the RING finger E3 ligase inhibitor is a small molecule comprising a selective binding element and a functional inhibitory element. In certain further embodiments, the selective binding element binds to the specificity domain of the RING finger E3 ligase. In certain embodiments, only the selective binding element binds to the specificity domain of the RING finger E3 ligase.

In yet other embodiments, the invention provides a method of designing a RING finger E3 ligase selective inhibitor, comprising a 3D structure analysis of the interaction of a binding partner of a RING finger E3 ligase with loop3 of the RING finger E3 ligase. In certain embodiments, the 3D structure is determined by homology modeling.

In certain embodiments of the invention, a selective inhibitor interacts with the specificity domain of a RING finger E3 ligase. In additional embodiments of the invention, a selective inhibitor selectively inhibits the interaction of a RING finger E3 ligase and a RING finger E3 ligase associated protein. In further embodiments, a selective inhibitor of the invention selectively inhibits the interaction of a RING finger E3 ligase with one E2 over another E2. In certain embodiments, a selective inhibitor of the invention selectively inhibits the ubiquitin ligase activity of a RING finger E3 ligase over another RING finger E3 ligase.

### BRIEF DESCRIPTION OF THE DRAWINGS

FIG. 1 is a schematic of a typical RING domain, showing eight conserved zinc-coordinating residues (either Cys or His, shown as solid circles containing an Arabic numeral) and the position of the coordinated metal (shown as gray circles containing a Roman numeral). Loop 1 occurs between coordinating residues 2 and 3. Loop 2 occurs between coordinating residues 4 and 5. Loop 3 occurs between coordinating residues 6 and 7.

FIG. 2 shows two views of the NMR model of a Brca1 RING domain and flanking helices (PDB: 1JM7 chain A). Cysteines are shown in black. Loops 1, 2 and 3 are indicated by labeled arrows, and loop 3 is circled.

FIG. 3 shows sequence alignments for loop 3 sequences of Group 1.

FIG. 4 shows sequence alignments for loop 3 sequences of Group 2.

FIG. 5 shows sequence alignments for loop 3 sequences of Group 3.

FIG. 6 is a graph depicting the selectivity of hPOSH ubiquitination inhibitors. Each hPOSH inhibitor was tested in triplicate incubations (at 3 μM) in parallel ubiquitination assays for hPOSH, hMdm2 and c-Cbl. Results are presented as the mean value of the activity measured in the individual experiments and are expressed as ubiquitination activity relative to the activity in the absence of compounds.

FIG. 7 is a graph depicting loop3 selectivity of hPOSH ubiquitination inhibitors. Each hPOSH inhibitor shown in the graph was tested in triplicate incubations (at 5 μM) in parallel ubiquitination assays for hPOSH, Cbl-b and hPOSH containing loop 3 of Cbl-b RING domain. Results are presented as the mean value of the activity measured in the individual experiments and are expressed as ubiquitination activity relative to the activity in the absence of compounds.

### DETAILED DESCRIPTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “binding” refers to a direct association between two molecules, due to, for example, covalent, electrostatic, hydrophobic, ionic and/or hydrogen-bond interactions under physiological conditions.

The terms “test compound” and “test agent” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

A “chimeric protein” or “fusion protein” is a fusion of a first amino acid sequence with a second amino acid sequence, wherein the first and second amino acid sequences are not naturally part of a single polypeptide.

The term “including” is used herein to mean, and is used interchangeably with, the phrase “including but not limited to”.

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The terms “proteins” and “polypeptides” are used interchangeably herein.

The term “or” is used herein to mean, and is used interchangeably with, the term “and/or”, unless context clearly indicates otherwise.

A “RING finger E3 ligase associated protein” or “RING finger E3 ligase-AP” refers to a protein capable of interacting with and/or binding to a RING finger E3 ligase polypeptide. Generally, the RING finger E3 ligase associated protein may interact directly or indirectly with the POSH polypeptide.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

2. Overview

The application relates in part to the discovery of a portion of E3 proteins that is unique or nearly unique for each E3. This domain is termed the “specificity domain”. E3 proteins interact with many different proteins, including E2 proteins, substrate proteins and proteins that help localize E3s to the appropriate cellular localizations. E3 proteins may also interact with regulatory proteins, such as kinases. Many E3 proteins contain related domains, such as RING domains, and E3s also have in common the ability to act as ubiquitin ligases. E3s tend to interact with ubiquitin as well. Altering the sequence of an E3 or causing an E3 to bind to a binding agent is likely to perturb one or more of these various interactions or activities and affect the biological function(s) that the E3 participates in.

E3 proteins, and the ubiquitination pathway generally, participate in a number of disease states, including hyperproliferative states, such as cervical and breast cancers, viral infections, such as HIV, human papilloma virus and Epstein-Barr virus infections, neurological disorders, such as Parkinson's disease, and inflammatory diseases. While it may be possible to design pharmaceutical agents that generally inhibit E3 activity, such agents may be highly pleiotropic in effects and may interfere with many processes that are preferably not perturbed. Specificity domains disclosed herein may be used, for example, to target agents at particular E3s, and preferably E3s that are involved in a particular disease state for which a therapeutic intervention is desired.

3. Methods for Predicting or Identifying a Specificity Domain

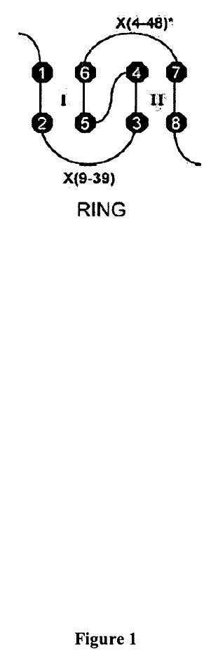

In certain embodiments, the application provides methods for predicting or identifying a specificity domain in an E3 polypeptide. In certain embodiments, a specificity domain may be identified by locating, in a known or suspected E3 polypeptide, a RING domain. A “RING domain” or “Ring Finger” is a zinc-binding domain with a defined octet of cysteine and histidine residues. Certain examples of RING domains comprise the consensus sequences as set forth below (Xaa indicates a non-conserved position): Cys Xaa Xaa Cys Xaa₉₋₃₉ Cys Xaa₁₋₃ His Xaa₂₋₃ Cys Xaa Xaa Cys Xaa₄₋₄₈ Cys Xaa Xaa Cys or Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys. In certain embodiments a RING domain is defined by a SMART Hidden Markov Model, using RPS BLAST. Various proteins with RING domains are defined in public databases, such as SMART and Pfam. Capilli et al. also describe methods for identifying RING domains. Capili A D, Schultz D C, RauscherIII F J, Borden K L. Solution structure of the PHD domain from the KAP-1 corepressor: structural determinants for PHD, RING and LIM zinc-binding domains. EMBO J. Jan. 15, 2001; 20(1-2):165-77. A diagram of a RING domain is shown in FIG. 1.

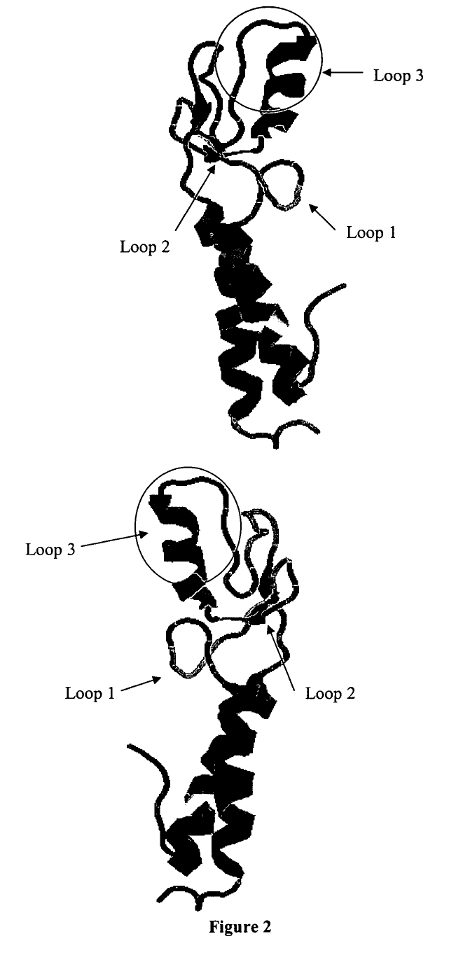

The specificity domain is situated between the 6^{th} and 7^{th} conserved metal-coordinating amino acids of the RING domain. Conserved metal-coordinating are generally Cys and His. The specificity domain may also be called “loop 3”, with “loop 2” being the sequence situated between the 4^{th} and 5^{th} metal-coordinating amino acids, and with “loop 1” being the sequence situated between the 2^{nd} and 3^{rd} metal-coordinating amino acids. Specificity domains of known human E3 proteins range in size from 4 to 48 amino acids, although it is understood that different sizes may be found in as yet undiscovered E3 proteins. In certain embodiments, a specificity domain is highly exposed on the outer surface of the E3, as shown for Brca1 in FIG. 2.

In certain embodiments, a plurality of specificity domains may be predicted or identified and assembled into a database for use, for example, in planning drug screening assays.

4. Specificity Domains

In certain aspects the application provides polypeptides consisting essentially of an E3 specificity domain. The term “consisting essentially of” is used in this respect to mean a polypeptide composed of a specificity domain and about 0 to 100 additional amino acids. The additional amino acids may be added at either the C-terminal or N-terminal end. In addition, a polypeptide consisting essentially of an E3 specificity domain may have one or more modifications, such as a hydrophobic modification, a phosphate, a biotin or other affinity purification label, a sugar moiety or a fluorescent moiety. Examples of E3 specificity domains are provided in Table 1, below.

In certain aspects the application provides fusion proteins comprising an E3 specificity domain and an additional amino acid sequence that is not normally present in a polypeptide with the E3 specificity domain. An E3 specificity domain may be fused to another polypeptide for a variety of purposes. For example, a fusion partner may be chosen to confer detectability, to attach the E3 specificity domain to a surface, to stabilize or retain the structure of an E3 specificity domain and/or to make purification easier. Examples of proteins to which an E3 specificity domain may be fused include green fluorescent protein (GFP) and variants thereof, glutathione-S-transferase (GST), polyhistidine (e.g. hexahistidine), and a heterologous E3 protein.

5. Protein-Protein Interactions and Drug Design

Characterization of the specificity of Chfr toward ubiquitin-conjugating enzymes has shown that both Ubc4 and Ubc5, but not UbcH7 and UbcH10, function with the Chfr ligase (Kang et al. (2002) J Cell Biol 156(2): 249-259). Several structural models of E2/E3 complexes are currently known. The UbcH5B/CNOT4 complex was revealed by combining NMR, mutagenesis and docking approaches (Dominguez et al. (2004) Structure Camb 12(4): 633-644). The structure of a c-Cbl/UbcH7 complex was characterized. The crystal structure of c-Cbl bound to a cognate E2 and a kinase peptide shows how the RING domain recruits the E2 (Zheng (2000) Cell 18:102(4): 533-539). The TRAF6 RING finger domain was shown to mediate physical interactions with Ubc13 (Wooff et al. (2004) FEBS Lett 566(1-3): 229-233).

Protein-protein interactions occur in a number of different ways. Antibody-antigen binding is one well-described protein-protein interaction. Protein-protein interactions also control the localization of proteins, their substrate-processing activity, and their tagging for destruction or recycling. A key feature of protein-protein interactions is their variety. Proteins interact in complicated ways because their shapes are so vastly complex. Amino acid side chains that stick out from the body of the molecule create pits or bumps of different shapes and sizes. Proteins exploit this structural diversity to the fullest, producing binding pockets and recognition sites with varying degrees of specificity and subtlety of interaction. It is this versatility of protein-protein interactions that makes them such a tempting prospect to exploit in the search for new drug targets.

Most current drugs target the important binding site of a protein, typically affecting its entire spectrum of operation. A new generation of drugs can act as competitive antagonists, but can also make much more subtle alterations through allosteric inhibition, by only disrupting the way in which a protein interacts with other specific proteins (Steve Buckingham (2004) Horizon Symposia April 2004). Molecules have been identified that allostericallly inhibit the function of inducible nitric oxide synthase by binding to the heme cofactor in the protein active site, which disrupts protein dimerization (Arkin et al. (2004) Natur Rev. Drug Discovery 3: 301-317). Recently, small-molecule inhibitors of the MDM2-p53 tumor suppressor protein interaction have been identified (Vassilev et al. (2004) Science 303(5659): 844-848). Furthermore, characterization of the p53 DNA binding domain was also achieved (Klein et al. (2004) Biol Chem 385(1): 95-102). Development of a binding assay for p53/HMDM2 by using Homogeneous Time-Resolved Fluorescence (HTRF) has been carried out by Merck Research Laboratories (Kane et al. (2000) Analytical Biochemistry 278: 29-38). A high throughput time-resolved fluorescence resonance energy transfer assay for TRAF6, a ubiquitin ligase involved in the Interleukin 1 receptor activation phathway, ubiquitin polymerization was also developed (Hong et al. (2003) Assay and drug development technologies 1(1-2): 175-180).

“Virtual screening” or “in silico screening” is the use of computational chemistry techniques to analyze large chemical databases in order to identify possible new drug candidates. Virtual screening techniques range from simple ones, based on the presence or absence of specific substructures, or match in calculated molecular properties, up to sophisticated virtual docking methods aimed at fitting putative ligand molecules into the target 3D structure. A review of current docking and scoring methods on systems of pharmaceutical relevance may be found in (Perola et al. (2004) Proteins 1;56(2): 235-249). A new approach for rapid, accurate docking and scoring as well as Enrichment factors in database screening are described in Halgren et al. (2004) J Med Chem 47(7): 1750-1759. Additional reviews on virtual screening methods that complement HTS my be found in Stahura & Bajorath. (2004) Comb Chem Hight Throughput Screen 7(4): 259-269 as well as in Hann & Oprea. (2004) Curr Opin Chem Biol 8(3):255-263. Structure-based generation of viable leads from small combinatorial libraries is broadly described in Laird & Blake. (2004) Curr Opin Drug Discov Devel 7(3): 354-359. Further description of pharmacophore design, characterization of binding regions and the structure-activity approach can be found in Funk et al. (2004) J Med Chem 47(11): 2750-2760; Cunningham et al. (2004) SAR QSAR Environ Res 15(1): 55-67; Sun et al. (2004) Bioorg Med Chem 12(10): 2671-2677; Hu et al. (2004) Biochem Biophy Res Commun 316(3): 698-704; and Gouldson et al. (2004) Proteins 56(1): 67-84.

Virtual docking programs for use in structure based drug design are readily available. Accelrys, for example, provides “Affinity” for docking of a flexible ligand to a protein active site (see http://www.accelrys.com/insight/affinity.html). Tripoz makes available “FlexX™”, which is a fast algorithm for flexibly docking small ligands, using incremental construction to build the ligands in the binding site (see http://www.tripos.com/SciTech/inSilicoDisc/virtualScreening/flexx.html). Additionally, OpenEye Scientific Software offers the docking program, “FRED” (see http://www.eyesopen.com/products/applications/fred.html).

6. Binding Agents and Assays Related to E3 Specificity Domains

In some aspects, the present application provides binding agents for E3 specificity domains. In certain embodiments, such binding agents for an E3 specificity domain may be used as therapeutic agents for human diseases. In some embodiments, the present invention provides methods of identifying binding agents for an E3 specificity domain.

A binding agent for an E3 specificity domain may be any molecule or complex of molecules which is capable of binding to an E3 specificity domain. Exemplary binding agents for E3 specificity domains may include, for example, antibodies, antibody fragments, peptides, polypeptides, peptidomimetics, aptamers and small molecules. The E3 specificity domain-binding agents having limited cross-reactivity among different E3 members are generally preferred.

One embodiment of the invention pertains to an antibody specifically reactive with an E3 specificity domain. For example, by using immunogens derived from an E3 specificity domain (e.g., based on the cDNA sequences), antisera or monoclonal antibodies can be made by standard protocols (See, for example, Antibodies: A Laboratory Manual ed. by Harlow and Lane (Cold Spring Harbor Press: 1988)). An immunogenic form of an E3 specificity domain (e.g., an antigenic fragment of an E3 specificity domain, a fusion protein containing an E3 specificity domain or an antigenic fragment thereof) which is capable of eliciting an antibody response can be used to immunize a mammal, such as a mouse, a hamster or rabbit. Accordingly, in certain embodiments, the present invention provides antigenic fragments of an E3 specificity domain and fusion proteins containing an E3 specificity domain or an antigenic fragment thereof, which are capable of eliciting an antibody response. A mammal, such as a mouse, a hamster or rabbit can be immunized with an immunogenic form of the peptide (e.g., an E3 specificity domain or an antigenic fragment thereof which is capable of eliciting an antibody response). Techniques for conferring immunogenicity on a protein or peptide include conjugation to carriers or other techniques well known in the art. An immunogenic portion of an E3 specificity domain can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of an E3 specificity domain, antisera or polyclonal antibodies (if desired) against an E3 specificity domain can be obtained. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256:495-497), the human B cell hybridoma technique (Kozbar et al., (1983) Immunology Today, 4:72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with an E3 specificity domain and monoclonal antibodies isolated from a culture comprising such hybridoma cells.

The term antibody as used herein is intended to include fragments thereof which are also specifically reactive with an E3 specificity domain. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for an E3 specificity domain conferred by at least one CDR region of the antibody. In preferred embodiments, the antibody further comprises a label attached thereto and able to be detected, (e.g., the label can be a radioisotope, fluorescent compound, enzyme or enzyme co-factor).

In certain embodiments, antibodies against E3 specificity domains can be used, e.g., to monitor levels of or to localize a specific E3 polypeptide in an individual. Another application of antibodies against E3 specificity domains can be used to identify and isolate substrates or other molecules that are associated with a specific E3 polypeptide, for example, in immunoprecipitation studies. Another application of antibodies against E3 specificity domains is in the immunological screening of cDNA libraries constructed in expression vectors such as gt11, gt18-23, ZAP, and ORF8. Messenger libraries of this type, having coding sequences inserted in the correct reading frame and orientation, can produce fusion proteins. Thus, alternate isoforms (including splice variants) from humans may be identified.

“Selectivity” of an agent of the invention refers to the ability of an agent, such as a small molecule inhibitor of a ubiquitin ligase, to preferentially interact with one protein over another. For example, in certain instances, an agent will bind to one protein and not bind to another. Also, for example, in other instances, an agent will bind to more than one protein, but will bind one protein more strongly than it binds another. In certain embodiments, an agent of the invention will inhibit one E3 and not inhibit another, different E3. For example, a RING finger E3 ligase inhibitor of the invention may inhibit the ubiquitin ligase activity of one RING finger E3 ligase but not inhibit the ubiquitin ligase activity of another, different RING finger E3 ligase. In additional embodiments, the invention relates to agents that inhibit the interaction of an E3 ligase with one E2 but do not inhibit the interaction of the same E3 ligase with a different E2. In certain embodiments, the invention relates to agents that inhibit the ubiquitin ligase activity of more than one E3 ligase but inhibit to a greater degree the ubiquitin ligase activity of one E3 over another, different E3. For example, an agent of the invention may inhibit entirely the ubiquitin ligase activity of one E3 but inhibit only partially or not at all the ubiquitin ligase activity of a different E3.

A selective inhibitor of the invention has selectivity for at least one polypeptide over another polypeptide. For example, in certain embodiments, a selective inhibitor of the invention may inhibit the ubiquitin ligase activity of two different E3 polypeptides, but selectively targets only one of them, resulting in selective inhibition of the ubiquitin ligase activity of the targeted E3 polypeptide.

Certain embodiments of the present invention relate to assays for identifying binding agents for an E3 specificity domain. A binding agent that selectively binds to one E3 specificity domain but not to another E3 specificity domain is preferred.

A wide variety of assays may be used for this purpose, including labeled in vitro protein-test agent binding assays, yeast two-hybrid assays, electrophoretic mobility shift assays, immunoassays for protein binding, and the like. The predicted or solved three-dimensional structure (e.g., crystal or solution structure) of a polypeptide comprising an E3 specificity domain may be used for modeling candidate agents that are likely to bind to the E3. In some embodiments, the assay detects binding agents that selectively modulate the biological activity of an E3 polypeptide, such as an enzymatic activity, interaction to other molecules or cellular components, cellular compartmentalization, and the like.

A variety of assay formats will suffice and, in light of the present disclosure, those not expressly described herein will nevertheless be comprehended by one of ordinary skill in the art. Simple binding assays may be used to detect binding agents for an E3 specificity domain. Such binding assays may also identify binding agents that act by selectively binding to an E3 specificity domain so that the association between an E3 polypeptide and its associated molecule (e.g., a substrate polypeptide) can be disrupted. Assay formats which approximate such conditions as formation of protein complexes, enzymatic activity, may be generated in many different forms, and include assays based on cell-free systems (e.g., purified proteins or cell lysates), as well as cell-based assays which utilize intact cells. These test binding agents for E3 specificity domains to be tested can be produced, for example, by bacteria, yeast or other organisms (e.g., natural products), produced chemically (e.g., small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

Often, it will be desirable to immobilize a polypeptide to facilitate separation of polypeptides bound to a test binding agent from unbound polypeptides, as well as to accommodate automation of the assay. In an illustrative embodiment, a fusion protein containing an E3 specificity domain can be provided which adds a domain that permits the fusion protein to be bound to an insoluble matrix. In a further embodiment, binding agents for an E3 specificity domain may be identified by using an immobilized E3 specificity domain.

In general, where the screening assay is a binding assay (whether protein-protein binding, agent-protein binding, etc.), one or more of the molecules may be joined to a label, where the label can directly or indirectly provide a detectable signal. Various labels include radioisotopes, fluorescers, chemiluminescers, enzymes, specific binding molecules, particles (e.g., magnetic particles), and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins (e.g., albumin), detergents, etc. that are used to facilitate optimal protein-protein binding and/or reduce nonspecific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4° and 40° C. Incubation periods are selected for optimum activity, but may also be optimized to facilitate rapid high-throughput screening.

In certain embodiments, the invention provides assays to identify, optimize or otherwise assess agents that increase or decrease a ubiquitin-related activity (e.g., ligase activity) of a RING finger E3 ligase polypeptide. Ubiquitin-related activities of RING finger E3 ligase polypeptides may include the self-ubiquitination activity of a RING finger E3 ligase polypeptide, generally involving the transfer of ubiquitin from an E2 enzyme to the RING finger E3 ligase polypeptide, and the ubiquitination of a target protein, generally involving the transfer of a ubiquitin from a RING finger E3 ligase polypeptide to the target protein. In certain embodiments, a RING finger E3 ligase activity is mediated, at least in part, by a RING finger E3 ligase RING domain.

In certain embodiments, an assay comprises forming a mixture comprising a RING finger E3 ligase (e.g., POSH, Cbl-b), an E2 polypeptide and a source of ubiquitin (which may be the E2 polypeptide pre-complexed with ubiquitin). Optionally the mixture comprises an E1 polypeptide and optionally the mixture comprises a target polypeptide. Additional components of the mixture may be selected to provide conditions consistent with the ubiquitination of the RING finger E3 ligase polypeptide. One or more of a variety of parameters may be detected, such as RING finger E3 ligase-ubiquitin conjugates, E2-ubiquitin thioesters, free ubiquitin and target polypeptide-ubiquitin complexes. The term “detect” is used herein to include a determination of the presence or absence of the subject of detection (e.g., RING finger E3 ligase-ubiquitin, E2-ubiquitin, etc.), a quantitative measure of the amount of the subject of detection, or a mathematical calculation of the presence, absence or amount of the subject of detection, based on the detection of other parameters. The term “detect” includes the situation wherein the subject of detection is determined to be absent or below the level of sensitivity. Detection may comprise detection of a label (e.g., fluorescent label, radioisotope label, and other described below), resolution and identification by size (e.g., SDS-PAGE, mass spectroscopy), purification and detection, and other methods that, in view of this specification, will be available to one of skill in the art. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used. In a preferred embodiment, an assay comprises detecting the POSH-ubiquitin conjugate.

In certain embodiments, an assay comprises forming a mixture comprising a RING finger E3 ligase polypeptide, a target polypeptide and a source of ubiquitin (which may be the RING finger E3 ligase polypeptide pre-complexed with ubiquitin). Optionally the mixture comprises an E1 and/or E2 polypeptide and optionally the mixture comprises an E2-ubiquitin thioester. Additional components of the mixture may be selected to provide conditions consistent with the ubiquitination of the target polypeptide. One or more of a variety of parameters may be detected, such as RING finger E3 ligase-ubiquitin conjugates and target polypeptide-ubiquitin conjugates. In a preferred embodiment, an assay comprises detecting the target polypeptide-ubiquitin conjugate. In another preferred embodiment, an assay comprises detecting the RING finger E3 ligase-ubiquitin conjugate.

An assay described above may be used in a screening assay to identify agents that modulate a ubiquitin-related activity of a RING finger E3 ligase polypeptide. A screening assay will generally involve adding a test agent to one of the above assays, or any other assay designed to assess a ubiquitin-related activity of a RING finger E3 ligase polypeptide. The parameter(s) detected in a screening assay may be compared to a suitable reference. A suitable reference may be an assay run previously, in parallel or later that omits the test agent. A suitable reference may also be an average of previous measurements in the absence of the test agent. In general the components of a screening assay mixture may be added in any order consistent with the overall activity to be assessed, but certain variations may be preferred. For example, in certain embodiments, it may be desirable to pre-incubate the test agent and the E3 (e.g., the RING finger E3 ligase polypeptide), followed by removing the test agent and addition of other components to complete the assay. In this manner, the effects of the agent solely on the RING finger E3 ligase polypeptide may be assessed.

In certain embodiments, an assay is performed in a high-throughput format. For example, one of the components of a mixture may be affixed to a solid substrate and one or more of the other components is labeled. For example, the RING finger E3 ligase polypeptide may be affixed to a surface, such as a 96-well plate, and the ubiquitin is in solution and labeled. An E2 and E1 are also in solution, and the RING finger E3 ligase-ubiquitin conjugate formation may be measured by washing the solid surface to remove uncomplexed labeled ubiquitin and detecting the ubiquitin that remains bound. Other variations may be used. For example, the amount of ubiquitin in solution may be detected. In certain embodiments, the formation of ubiquitin complexes may be measured by an interactive technique, such as FRET, wherein a ubiquitin is labeled with a first label and the desired complex partner (e.g., RING finger E3 ligase polypeptide or target polypeptide) is labeled with a second label, wherein the first and second label interact when they come into close proximity to produce an altered signal. In FRET, the first and second labels are fluorophores. FRET is described in greater detail below. The formation of polyubiquitin complexes may be performed by mixing two or more pools of differentially labeled ubiquitin that interact upon formation of a polyubiqutin (see, e.g., US Patent Publication 20020042083). High-throughput may be achieved by performing an interactive assay, such as FRET, in solution as well. In addition, if a polypeptide in the mixture, such as the RING finger E3 ligase polypeptide or target polypeptide, is readily purifiable (e.g., with a specific antibody or via a tag such as biotin, FLAG, polyhistidine, etc.), the reaction may be performed in solution and the tagged polypeptide rapidly isolated, along with any polypeptides, such as ubiquitin, that are associated with the tagged polypeptide. Proteins may also be resolved by SDS-PAGE for detection.

In certain embodiments, the ubiquitin is labeled, either directly or indirectly. This typically allows for easy and rapid detection and measurement of ligated ubiquitin, making the assay useful for high-throughput screening applications. As descrived above, certain embodiments may employ one or more tagged or labeled proteins. A “tag” is meant to include moieties that facilitate rapid isolation of the tagged polypeptide. A tag may be used to facilitate attachment of a polypeptide to a surface. A “label” is meant to include moieties that facilitate rapid detection of the labeled polypeptide. Certain moieties may be used both as a label and a tag (e.g., epitope tags that are readily purified and detected with a well-characterized antibody). Biotinylation of polypeptides is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

An “E1” is a ubiquitin activating enzyme. In a preferred embodiment, E1 is capable of transferring ubiquitin to an E2. In a preferred embodiment, E1 forms a high energy thiolester bond with ubiquitin, thereby “activating” the ubiquitin. An “E2” is a ubiquitin carrier enzyme (also known as a ubiquitin conjugating enzyme). In a preferred embodiment, ubiquitin is transferred from E1 to E2. In a preferred embodiment, the transfer results in a thiolester bond formed between E2 and ubiquitin. In a preferred embodiment, E2 is capable of transferring ubiquitin to a RING finger E3 ligase polypeptide.

In an alternative embodiment, a RING finger E3 ligase polypeptide, E2 or target polypeptide is bound to a bead, optionally with the assistance of a tag. Following ligation, the beads may be separated from the unbound ubiquitin and the bound ubiquitin measured. In a preferred embodiment, RING finger E3 ligase polypeptide is bound to beads and the composition used includes labeled ubiquitin. In this embodiment, the beads with bound ubiquitin may be separated using a fluorescence-activated cell sorting (FACS) machine. Methods for such use are described in U.S. patent application Ser. No. 09/047,119, which is hereby incorporated in its entirety. The amount of bound ubiquitin can then be measured.

In a screening assay, the effect of a test agent may be assessed by, for example, assessing the effect of the test agent on kinetics, steady-state and/or endpoint of the reaction.

The components of the various assay mixtures provided herein may be combined in varying amounts. In a preferred embodiment, ubiquitin (or E2 complexed ubiquitin) is combined at a final concentration of from 5 to 200 ng per 100 microliter reaction solution. Optionally E1 is used at a final concentration of from 1 to 50 ng per 100 microliter reaction solution. Optionally E2 is combined at a final concentration of 10 to 100 ng per 100 microliter reaction solution, more preferably 10-50 ng per 100 microliter reaction solution. In a preferred embodiment, RING finger E3 ligase polypeptide is combined at a final concentration of from 1 to 500 ng per 100 microliter reaction solution.

Generally, an assay mixture is prepared so as to favor ubiquitin ligase activity and/or ubiquitination activity. Generally, this will be physiological conditions, such as 50-200 mM salt (e.g., NaCl, KCl), pH of between 5 and 9, and preferably between 6 and 8. Such conditions may be optimized through trial and error. Incubations may be performed at any temperature which facilitates optimal activity, typically between 4 and 40° C. Incubation periods are selected for optimum activity, but may also be optimized to facilitate rapid high through put screening. Typically between 0.5 and 1.5 hours will be sufficient. A variety of other reagents may be included in the compositions. These include reagents like salts, solvents, buffers, neutral proteins, e.g., albumin, detergents, etc. which may be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine tri-phosphate (ATP). The mixture of components may be added in any order that promotes ubiquitin ligase activity or optimizes identification of candidate modulator effects. In a preferred embodiment, ubiquitin is provided in a reaction buffer solution, followed by addition of the ubiquitination enzymes. In an alternate preferred embodiment, ubiquitin is provided in a reaction buffer solution, a candidate modulator is then added, followed by addition of the ubiquitination enzymes.

In general, a test agent that decreases a RING finger E3 ligase ubiquitin-related activity may be used to inhibit RING finger E3 ligase function in vivo, while a test agent that increases a RING finger E3 ligase ubiquitin-related activity may be used to stimulate RING finger E3 ligase function in vivo. Test agents may be modified for use in vivo, e.g., by addition of a hydrophobic moiety, such as an ester.

Certain embodiments of the application relate to assays for identifying agents that bind to an E3 specificity domain. A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions and design of test agents. In one embodiment, an assay detects agents which inhibit interaction of one or more subject RING finger E3 ligase polypeptides with a RING finger E3 ligase-AP. In another embodiment, the assay detects agents which modulate the intrinsic biological activity of a RING finger E3 ligase polypeptide or RING finger E3 ligase complex, such as an enzymatic activity, binding to other cellular components, and the like.

In one aspect, the application provides methods and compositions for the identification of compositions that interfere with the function of RING finger E3 ligase.

Assaying RING finger E3 ligase complexes, in the presence and absence of a candidate inhibitor, can be accomplished in any vessel suitable for containing the reactants. Examples include microtitre plates, test tubes, and micro-centrifuge tubes.

In one embodiment of the present application, drug screening assays can be generated which detect inhibitory agents on the basis of their ability to interfere with assembly or stability of the RING finger E3 ligase complex. In an exemplary binding assay, the compound of interest is contacted with a mixture comprising a RING finger E3 ligase polypeptide and at least one interacting polypeptide. Detection and quantification of RING finger E3 ligase complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between the RING finger E3 ligase polypeptides and a substrate polypeptide may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction

Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-RING finger E3 ligase fusion proteins can be adsorbed onto glutathione sepharose beads (Sigma Chemical, St. Louis, Mo.) or glutathione derivatized microtitre plates, which are then combined with a potential interacting protein, e.g., an ³⁵S-labeled polypeptide, and the test compound and incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In a further embodiment, agents that bind to a RING finger E3 ligase or RING finger E3 ligase-AP may be identified by using an immobilized RING finger E3 ligase or RING finger E3 ligase-AP. In an illustrative embodiment, a fusion protein can be provided which adds a domain that permits the protein to be bound to an insoluble matrix. For example, GST-RING finger E3 ligase fusion proteins can be adsorbed onto glutathione sepharose beads (Sigma Chemical, St. Louis, Mo.) or glutathione derivatized microtitre plates, which are then combined with a potential labeled binding agent and incubated under conditions conducive to binding. Following incubation, the beads are washed to remove any unbound agent, and the matrix bead-bound label determined directly, or in the supernatant after the bound agent is dissociated.

In yet another embodiment, the RING finger E3 ligase polypeptide and potential interacting polypeptide can be used to generate an interaction trap assay (see also, U.S. Pat. No.: 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., a RING finger E3 ligase polypeptide of sufficient length to bind to a potential interacting protein. The second hybrid protein encodes a transcriptional activation domain fused in frame to a gene encoding a “fish” protein, e.g., a potential interacting protein of sufficient length to interact with the RING finger E3 ligase polypeptide portion of the bait fusion protein. If the bait and fish proteins are able to interact, e.g., form a RING finger E3 ligase complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In still further embodiments of the present assay, the RING finger E3 ligase complex is generated in whole cells, taking advantage of cell culture techniques to support the subject assay. For example, as described below, the RING finger E3 ligase complex can be constituted in a eukaryotic cell culture system, including mammalian and yeast cells. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the RING finger E3 ligase complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In further embodiments, the application provides methods for identifying targets for therapeutic intervention. A polypeptide that interacts with a RING finger E3 ligase or participates in a RING finger E3 ligase-mediated process may be used to identify candidate therapeutics. Such targets may be identified by identifying proteins that associated with a RING finger E3 ligase (RING finger E3 ligase-APs) by, for example, immunoprecipitation with an anti-RING finger E3 ligase antibody, in silico analysis of high-throughput binding data, two-hybrid screens, and other protein-protein interaction assays described herein or otherwise known in the art in view of this disclosure. Agents that bind to such targets or disrupt protein-protein interactions thereof, or inhibit a biochemical activity thereof may be used in such an assay.

### Exemplification

The invention now being generally described, it will be more readily understood by reference to the following examples, which are included merely for purposes of illustration of certain aspects and embodiments of the present invention, and are not intended to limit the invention.

### EXAMPLE 1

The following analysis demonstrates the high level of sequence variability found in the Loop 3 of the human RING domain proteins.

A representative list of human RING domains was constructed as follows:

(1) Search the human proteome for RING domains as defined by SMART Hidden Markov Model (HMM), using RPS blast. The RING domain was found using the SMART and Pfam databases, through the RPS-Blast program.

(2) Search for regular sequence properties within the SMART RING domain, and cut the sequence to take the sequence portion from the first metal coordinating residue to the eighth metal coordinating residue.

(3) Remove redundant domains.

(4) Use CLUSTALW to produce a multiple alignment of the RING domains.

(5) Use a scoring matrix to calculate the extent of conservation (identity) of all collected RING domains at each residue.

For the purposes of comparing loop 3 variability, the E3s were categorized based on the length of the loop 3.

Group 1: 34 sequences with loop3 length of 13 aa.

Group 2: 27 sequences with loop3 length of 11 aa.

Group 3: 23 sequences with loop3 length of 10 aa.

Each group of sequences was aligned using the ClustalW algorithm.

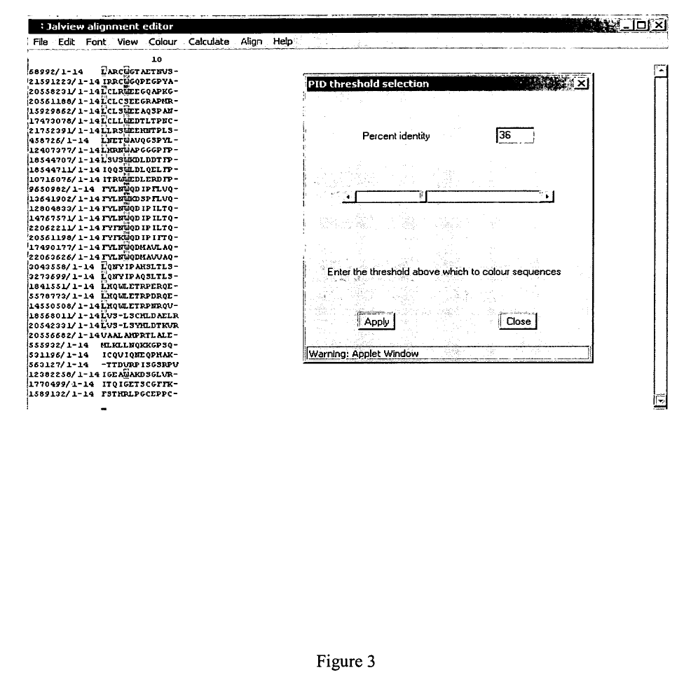

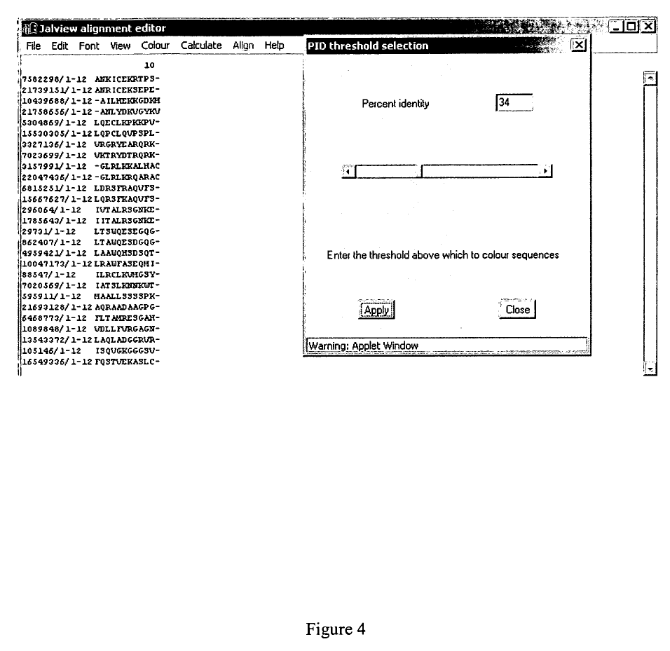

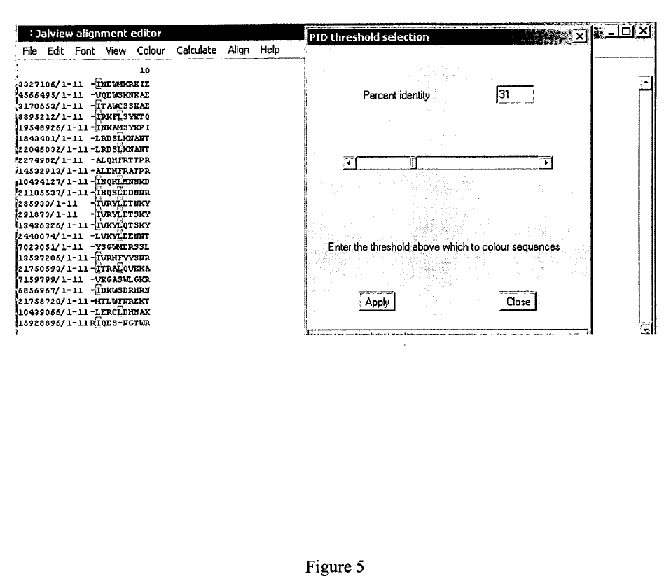

The aligned sequences were analyzed using the PID threshold selection from the JalView program and are illustrated in FIGS. 3-5. The loop3 sequences used for this comparison are shown in Table 1.

By analyzing loop3 from the RING domains, one can see that loop3 is highly variable, in all six groups. By looking at identity between 27% and 36% in all the sequences, there are only 0-2 conserved amino acids.

Group 1-Short:

At 36% identity between all the sequences, there are only two amino acids that are conserved.

Group 2-Short:

At 34% identity between all the sequences, there are no amino acids that are conserved.

Group 3-Short:

At 31% identity between all the sequences, there are only two amino acids that are conserved.

TABLE 1

Examples of E3 Specificity Domains

| Group 1 sequences: | Group 2 sequences: | Group 3 sequences: |
|---|---|---|
| (SEQ ID Nos. 1-34) | (SEQ ID Nos. 35-61) | (SEQ ID Nos. 62-84) |
| >68992 | >29731 | >6856967 |
| LARCWGTAETNVS | LTSWQESEGQG | IDKWSDRHRN |
| >458726 | >88547 | >2274982 |
| LNETWAVQGSPYL | ILRCLKVMGSY | ALQHFRTTPR |
| >531196 | >105146 | >3170653 |
| ICQVIQNEQPHAK | ISQVGKGGGSV | ITAWCSSKAE |
| >555932 | >595911 | >3327106 |
| MLKLLNQKKGPSQ | MAALLSSSSPK | INEWMKRKIE |
| >563127 | >862407 | >7159799 |
| TTDVRPISGSRPV | LTAWQESDGQG | VKGASWLGKR |
| >1589132 | >3157991 | >10434127 |
| FSTHRLPGCEPPC | GLRLKKALHAC | INQHLMNNKD |
| >1770499 | >3327136 | >10439066 |
| ITQIGETSCGFFK | VRGRYEARQRK | LERCLDHNAK |
| >1841551 | >4959421 | >14532913 |
| LHQWLETRPERQE | LAAWQHSDSQT | ALEHFRATPR |
| >3043558 | >5304869 | >21105537 |
| LQNYIPAHSLTLS | LQECLKPKKPV | IHQSLEDNNR |
| >3273699 | >6815251 | >21758720 |
| LQNYIPAQSLTLS | LDRSFRAQVFS | MTLWFNREKT |
| >5578773 | >7020569 | >285933 |
| LHQWLETRPDRQE | IATSLKNNKWT | IVRYLETNKY |
| >9650982 | >7023699 | >291873 |
| FYLNWQDIPFLVQ | VKTRYDTRQRK | IVRYLETSKY |
| >10716076 | >7582298 | >2440074 |
| ITRWWEDLERDFP | ANKICEKRTPS | LVKYLEENNT |
| >12382258 | >10047173 | >4566495 |
| IGEAWAKDSGLVR | LRAWFASEQMI | VQEWSKNKAE |
| >12407377 | >13543372 | |
| TQ | ANRICEKSEPE | INKANSYKPI |
| >15929862 | >22047436 | >21750593 |
| LCLSWEEAQSPAN | GLRLKRQARAC | ITRALQVKKA |
| >17473078 | >296064 | >15928896 |
| LCLLWEDTLTPNC | IVTALRSGNKE | RIQESNGTWR |
| >17490177 | >1785643 | >1843401 |
| FYLNWQDMAVLAQ | IITALRSGNKE | LRDSLKNANT |
| >18544707 | >6468773 | >22046032 |
| LSVSWKDLDDTFP | FLTAMRESGAH | LRDSLKN |
| >18544711 | >10439688 | |
| IQQSWLDLQELFP | AILHEKK | |
| >18568011 | >15530305 | |
| LVSLSCHLDAELR | LQPCLQV | |
| >20542331 | >21758656 | |
| LVSLSYHLDTKVR | ANLYDKVG | |
| >20558231 | >1089848 | |
| LCLRWEEGQAPKG | VDLLFVRGAGN | |
| >20561188 | | |
| LCLCSEEGRAPMR | | |
| >20561198 | | |
| FYFKWQDIPIFTQ | | |
| >21591223 | | |
| IRRCWGQPEGPYA | | |
| >21752391 | | |
| LLRSWEEHNTPLS | | |
| >22062211 | | |
| FYFNWQDIPILTQ | | |
| >22063626 | | |
| FYLNWQDMAVVAQ | | |
| >20556682 | | |
| VAALAHPRTLALE | | |

### EXAMPLE 2

Construction and Purification of POSH, Cbl-b and Loop-3 Chimeric RING Domains.

Glutathione S-transferase (GST) fusion plasmids were constructed by PCR amplification of hPOSH codons 1-139 and Cbl-b codons 1-490. The amplified PCR products were then individually cloned into pGEX-6P-2 (Amersham Pharmacia Biotech, Buckinghamshire, UK). hPOSH^{Loop3(Cbl-b)} contains hPOSH codons 1-139 where codons 37-48 are replaced with codons 397-407 from Cbl-b. Cbl-b^{Loop3(hPOSH)} contains Cbl-b codons 1-490 where codons 397-407 are replaced with codons 37-48 from hPOSH. Construction of the chimeric RING constructs was done by PCR mutagenesis of pGEX-hPOSH (1-139) and pGEX-Cbl-b (1-490), using overlapping primers containing the region to be replaced. Sense primers were used together with a 3′ vector derived primer and complementary primers were used together with 5′ vector derived primer to first amplify 3′ and 5′ portions of chimeric product, respectively. PCR products from these reactions were purified, 3′ and 5′ overlapping products were mixed together and amplified with the vector derived 3′ and 5′ primers to amplify a complete chimeric PCR product, which was digested with the appropriate restriction enzymes and ligated into pGEX-6P-2.

The GST-hPOSH¹⁻¹³⁹, GST-hPOSH^{loop3(cblB)} and GST-Cbl-b¹⁻⁴⁹⁰ and GST-Cbl-b^{loop3(hPOSH)} were generated in E. coli BL21. Bacterial cultures were grown in LB media with carbenicillin (100 μg/ml) and recombinant protein production was induced with 0.5 or 1 mM IPTG for 4 hours at 25 or 30° C. Cells were lysed by sonication and the recombinant proteins were then isolated from the cleared bacterial lysate by affinity chromatography on a glutathione-sepharose resin (Amersham Pharmacia Biotech, Buckinghamshire, UK).

In all sequences below, hPOSH sequence is highlighted dark gray and Cbl-b sequence is highlighted light gray.

PCR primers

| | |
|---|---|
| GST fusion | |
| Protein | Primers (sense/complementary) |

| | | |
|---|---|---|
| hPOSH¹⁻¹³⁹ | 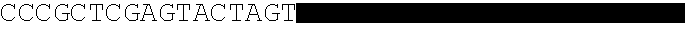 | (SEQ ID NO:85) |
| | 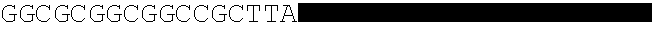 | (SEQ ID NO:86) |
| Cbl-b¹⁻⁴⁹⁰ | ATTACCCGGGATGGCAAACTCAATGAATGG | (SEQ ID NO:87) |
| | GGGCTCGAGTCTTCTCTGGGCAAGGGGA | (SEQ ID NO:88) |
| hPOSH^{Loop3(Cbl-b)} | 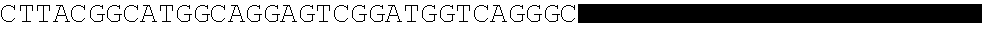 | (SEQ ID NO:89) |
| | 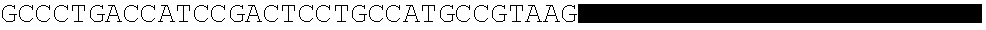 | (SEQ ID NO:90) |
| Cbl-b^{Loop3(hPosH)} | 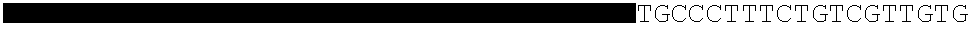 | (SEQ ID NO:91) |
| | 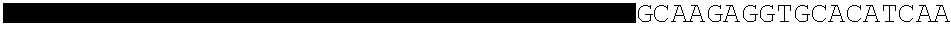 | (SEQ ID NO:92) |

DNA and protein sequences:

GST-hPOSH¹⁻¹³⁹
DNA sequence (SEQ ID NO: 93):

| | |
|---|---|
| 1 | ATG TCC CCT ATA CTA GGT TAT TGG AAA ATT AAG GGC CTT GTG |
| 43 | CAA CCC ACT CGA CTT CTT TTG GAA TAT CTT GAA GAA AAA TAT |
| 85 | GAA GAG CAT TTG TAT GAG CGC GAT GAA GGT GAT AAA TGG CGA |
| 127 | AAC AAA AAG TTT GAA TTG GGT TTG GAG TTT CCC AAT CTT CCT |
| 169 | TAT TAT ATT GAT GGT GAT GTT AAA TTA AdA CAG TCT ATG GCC |
| 211 | ATC ATA CGT TAT ATA GCT GAC AAG CAC AAC ATG TTG GGT GGT |
| 253 | TGT CCA AAA GAG CGT GCA GAG ATT TCA ATG CTT GAA GGA GCG |
| 295 | GTT TTG GAT ATT AGA TAC GGT GTT TCG AGA ATT GCA TAT AGT |
| 337 | AAA GAC TTT GAA ACT CTC AAA GTT GAT TTT CTT AGC AAG CTA |
| 379 | CCT GAA ATG CTG AAA ATG TTC GAA GAT CGT TTA TGT CAT AAA |
| 421 | ACA TAT TTA AAT GGT GAT CAT GTA ACC CAT CCT GAC TTC ATG |
| 463 | TTG TAT GAC GCT CTT GAT GTT GTT TTA TAC ATG GAC CCA ATG |
| 505 | TGC CTG GAT GCG TTC CCA AAA TTA GTT TGT TTT AAA AAA CGT |
| 547 | ATT GAA GCT ATC CCA CAA ATT GAT AAG TAC TTG AAA TCC AGC |
| 589 | AAG TAT ATA GCA TGG CCT TTG CAG GGC TGG CAA GCC ACG TTT |
| 631 | GGT GGT GGC GAC CAT CCT CCA AAA TCG GAT CTG GAA GTT CTG |
| 673 | TTC CAG GGG CCC CTG GGA TCC CCA GGA ATT CCC GGG TCG AGT |

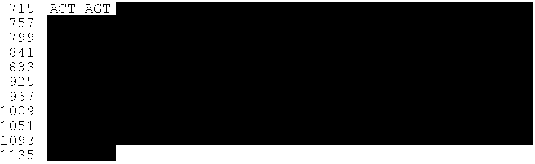

Protein sequence (SEQ ID NO: 94):

| | |
|---|---|
| 1 | MSPILGYWKI KGLVQPTRLL LEYLEEKYEE HLYERDEGDK WRNKKFELGL |
| 51 | EFPNLPYYID GDVKLTQSMA IIRYIADKHN MLGGCPKEPA EISMLEGAVL |
| 101 | DIRYGVSRIA YSKDFETLKV DFLSKLPEML KMFEDRLCHK TYLNGDHVTH |
| 151 | PDFMLYDALD VVLYMDPMCL DAFPKLVCFK KRIEAIPQID KYLKSSKYIA |

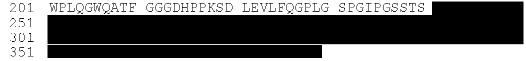

GST-Cbl-b¹⁻⁴⁹⁰
DNA sequence (SEQ ID NO: 95):

| | |
|---|---|
| 1 | ATG TCC CCT ATA CTA GGT TAT TGG AAA ATT AAG GGC CTT GTG |
| 43 | CAA CCC ACT CGA CTT CTT TTG GAA TAT CTT GAA GAA AAA TAT |
| 85 | GAA GAG CAT TTG TAT GAG CGC GAT GAA GGT GAT AAA TGG CGA |
| 127 | AAC AAA AAG TTT GAA TTG GGT TTG GAG TTT CCC AAT CTT CCT |
| 169 | TAT TAT ATT GAT GGT GAT GTT AAA TTA ACA CAG TCT ATG GCC |
| 211 | ATC ATA CGT TAT ATA GCT GAC AAG CAC AAC ATG TTG GGT GGT |
| 253 | TGT CCA AAA GAG CGT GCA GAG ATT TCA ATG CTT GAA GGA GCG |
| 295 | GTT TTG GAT ATT AGA TAC GGT GTT TCG AGA ATT GCA TAT AGT |
| 337 | AAA GAC TTT GAA ACT CTC AAA GTT GAT TTT CTT AGC AAG CTA |
| 379 | CCT GAA ATG CTG AAA ATG TTC GAA GAT CGT TTA TGT CAT AAA |
| 421 | ACA TAT TTA AAT GGT GAT CAT GTA ACC CAT CCT GAC TTC ATG |
| 463 | TTG TAT GAC GCT CTT GAT GTT GTT TTA TAC ATG GAC CCA ATG |
| 505 | TGC CTG GAT GCG TTC CCA AAA TTA GTT TGT TTT AAA AAA CGT |
| 547 | ATT GAA GCT ATC CCA CAA ATT GAT AAG TAC TTG AAA TCC AGC |
| 589 | AAG TAT ATA GCA TGG CCT TTG CAG GGC TGG CAA GCC ACG TTT |
| 631 | GGT GGT GGC GAC CAT CCT CCA AAA TCG GAT CTG GAA GTT CTG |
| 673 | TTC CAG GGG CCC CTG GGA TCC CCA GGA ATT CCC GGG ATG GCA |
| 715 | AAC TCA ATG AAT GGC AGA AAC CCT GGT GGT CGA GGA GGA AAT |
| 757 | CCC CGA AAA GGT CGA ATT TTG GGT ATT ATT GAT GCT ATT CAG |
| 799 | GAT GCA GTT GGA CCC CCT AAG CAA GCT GCC GCA GAT CGC AGG |
| 841 | ACC GTG GAG AAG ACT TGG AAG CTC ATG GAC AAA GTG GTA AGA |
| 883 | CTG TGC CAA AAT CCC AAA CTT CAG TTG AAA AAT AGC CCA CCA |
| 925 | TAT ATA CTT GAT ATT TTG CCT GAT ACA TAT CAG CAT TTA CGA |
| 967 | CTT ATA TTG AGT AAA TAT GAT GAC AAC CAG AAA CTT GCC CAA |
| 1009 | CTC AGT GAG AAT GAG TAC TTT AAA ATC TAC ATT GAT AGC CTT |
| 1051 | ATG AAA AAG TCA AAA CGG GCA ATA AGA CTC TTT AAA GAA GGC |
| 1093 | AAG GAG AGA ATG TAT GAA GAA CAG TCA CAG GAC AGA CGA AAT |
| 1135 | CTC ACA AAA CTG TCC CTT ATC TTC AGT CAC ATG CTG GCA GAA |
| 1177 | ATC AAA GCA ATC TTT CCC AAT GGT CAA TTC CAG GGA GAT AAC |
| 1219 | TTT CGT ATC ACA AAA GCA GAT GCT GCT GAA TTC TGG AGA AAG |
| 1261 | TTT TTT GGA GAC AAA ACT ATC GTA CCA TGG AAA GTA TTC AGA |
| 1303 | CAG TGC CTT CAT GAG GTC CAC CAG ATT AGC TCT GGC CTG GAA |
| 1345 | GCA ATG GCT CTA AAA TCA ACA ATT GAT TTA ACT TGC AAT GAT |
| 1387 | TAC ATT TCA GTT TTT GAA TTT GAT ATT TTT ACC AGG CTG TTT |
| 1429 | CAG CCT TGG GGC TCT ATT TTG CGG AAT TGG AAT TTC TTA GCT |
| 1471 | GTG ACA CAT CCA GGT TAC ATG GCA TTT CTC ACA TAT GAT GAA |
| 1513 | GTT AAA GCA CGA CTA CAG AAA TAT AGC ACC AAA CCC GGA AGC |
| 1555 | TAT ATT TTC CGG TTA AGT TGC ACT CGA TTG GGA CAG TGG GCC |
| 1597 | ATT GGC TAT GTG ACT GGG GAT GGG AAT ATC TTA CAG ACC ATA |
| 1639 | CCT CAT AAC AAG CCC TTA TTT CAA GCC CTG ATT GAT GGC AGC |
| 1681 | AGG GAA GGA TTT TAT CTT TAT CCT GAT GGG AGG AGT TAT AAT |
| 1723 | CCT GAT TTA ACT GGA TTA TGT GAA CCT ACA CCT CAT GAC CAT |
| 1765 | ATA AAA GTT ACA CAG GAA CAA TAT GAA TTA TAT TGT GAA ATG |
| 1807 | GGC TCC ACT TTT CAG CTC TGT AAG ATT TGT GCA GAG AAT GAC |
| 1849 | AAA GAT GTC AAG ATT GAG CCT TGT GGG CAT TTG ATG TGC ACC |
| 1891 | TCT TGC CTT ACG GCA TGG CAG GAG TCG GAT GGT CAG GGC TGC |
| 1933 | CCT TTC TGT CGT TGT GAA ATA AAA GGA ACT GAG CCC ATA ATC |
| 1975 | GTG GAT CCC TTT GAT CCA AGA GAT GAA GGC TCC AGG TGT TGC |
| 2017 | AGC ATC ATT GAC CCC TTT GGC ATG CCG ATG CTC GAC TTG GAC |
| 2059 | GAC GAT GAT GAT CGT GAG GAG TCC TTG ATG ATG AAT CGG TTG |
| 2101 | GCA AAC GTC CGA AAG TGC ACT GAC AGG CAG AAC TCA CCA GTC |
| 2143 | ACA TCA CCA GGA TCC TCT CCC CTT GCC CAG AGA AGA CTC GAG |
| 2185 | CGG CCG CAT CGT GAC TGA |

Protein sequence (SEQ ID NO: 96):

| | |
|---|---|
| 1 | MSPILGYWKI KGLVQPTRLL LEYLEEKYEE HLYERDEGDK WRNKKFELGL |
| 51 | EFPNLPYYID GDVKLTQSMA IIRYIADKHN MLGGCPKERA EISMLEGAVL |
| 101 | DIRYGVSRIA YSKDFETLKV DFLSKLPEML KMFEDRLCHK TYLNGDHVTH |
| 151 | PDFMLYDALD VVLYMDPMCL DAFPKLVCFK KRIEAIPQID KYLKSSKYIA |
| 201 | WPLQGWQATF GGGDHPPKSD LEVLFQGPLG SPGIPGMANS MNGRNPGGRG |
| 251 | GNPRKGRILG IIDAIQDAVG PPKQAAADRR TVEKTWKLMD KVVRLCQNPK |
| 301 | LQLKNSPPYI LDTLPDTYQH LRLILSKYDD NQKLAQLSEN EYFKIYIDSL |
| 351 | MKKSKRAIRL FKEGKERMYE EQSQDRRNLT KLSLIFSHML AEIKAIFPNG |
| 401 | QFQGDNFRIT KADAAEFWRK FFGDKTIVPW KVFRQCLHEV HQISSGLEAM |
| 451 | ALKSTIDLTC NDYISVFEFD IFTRLFQPWG SILRNWNFLA VTHPGYMAFL |
| 501 | TYDEVKARLQ KYSTKPGSYI FRLSCTRLGQ WAIGYVTGDG NILQTIPHNK |
| 551 | PLFQALIDGS REGFYLYPDG RSYNPDLTGL CEPTPHDHIK VTQEQYELYC |
| 601 | EMGSTFQLCK ICAENDKDVK IEPCGHLMCT SCLTAWQESD GQGCPFCRCE |
| 651 | IKGTEPIIVD PFDPRDEGSR CCSIIDPFGM PMLDLDDDDD REESLMMNRL |
| 701 | ANVRKCTDRQ NSPVTSPGSS PLAQRRLERP HRD |

GST-hPOSH^{Loop3(Cbl-b)}
DNA sequence (SEQ ID NO: 97):

| | |
|---|---|
| 1 | ATG TCC CCT ATA CTA GGT TAT TGG AAA ATT AAG GGC CTT GTG |
| 43 | CAA CCC ACT CGA CTT CTT TTG GAA TAT CTT GAA GAA AAA TAT |
| 85 | GAA GAG CAT TTG TAT GAG CGC GAT GAA GGT GAT AAA TGG CGA |
| 127 | AAC AAA AAG TTT GAA TTG GGT TTG GAG TTT CCC AAT CTT CCT |
| 169 | TAT TAT ATT GAT GGT GAT GTT AAA TTA ACA CAG TCT ATG GCC |
| 211 | ATC ATA CGT TAT ATA GCT GAC AAG CAC AAC ATG TTG GGT GGT |
| 253 | TGT CCA AAA GAG CGT GCA GAG ATT TCA ATG CTT GAA GGA GCG |
| 295 | GTT TTG GAT ATT AGA TAC GGT GTT TCG AGA ATT GCA TAT AGT |
| 337 | AAA GAC TTT GAA ACT CTC AAA GTT GAT TTT CTT AGC AAG CTA |
| 379 | CCT GAA ATG CTG AAA ATG TTC GAA GAT CGT TTA TGT CAT AAA |
| 421 | ACA TAT TTA AAT GGT GAT CAT GTA ACC CAT CCT GAC TTC ATG |
| 463 | TTG TAT GAC GCT CTT GAT GTT GTT TTA TAC ATG GAC CCA ATG |
| 505 | TGC CTG GAT GCG TTC CCA AAA TTA GTT TGT TTT AAA AAA CGT |
| 547 | ATT GAA GCT ATC CCA CAA ATT GAT AAG TAC TTG AAA TCC AGC |
| 589 | AAG TAT ATA GCA TGG CCT TTG CAG GGC TGG CAA GCC ACG TTT |
| 631 | GGT GGT GGC GAC CAT CCT CCA AAA TCG GAT CTG GAA GTT CTG |
| 673 | TTC CAG GGG CCC CTG GGA TCC CCA GGA ATT CCC GGG TCG AGT |

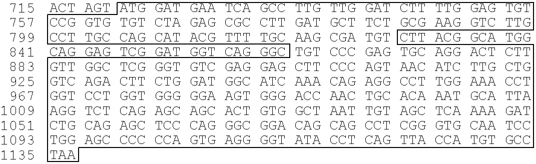

Protein sequence (SEQ ID NO: 98):

| | |
|---|---|
| 1 | MSPILGYWKI KGLVQPTRLL LEYLEEKYEE HLYERDEGDK WRNKKFELGL |
| 51 | EFPNLPYYID GDVKLTQSMA IIRYIADKHN MLGGCPKERA EISMLEGAVL |
| 101 | DIRYGVSRIA YSKDFETLKV DFLSKLPEML KMFEDRLCHK TYLNGDHVTH |
| 151 | PDFMLYDALD VVLYMDPMCL DAFPKLVCFK KRIEAIPQID KYLKSSKYIA |

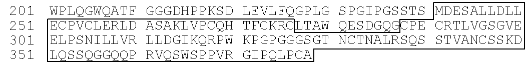

Cbl-b^{Loop3(hPosH)}
DNA sequence (SEQ ID NO: 99):

| | |
|---|---|
| 1 | ATG TCC CCT ATA CTA GGT TAT TGG AAA ATT AAG GGC CTT GTG |
| 43 | CAA CCC ACT CGA CTT CTT TTG GAA TAT CTT GAA GAA AAA TAT |
| 85 | GAA GAG CAT TTG TAT GAG CGC GAT GAA GGT GAT AAA TGG CGA |
| 127 | AAC AAA AAG TTT GAA TTG GGT TTG GAG TTT CCC AAT CTT CCT |
| 169 | TAT TAT ATT GAT GGT GAT GTT AAA TTA ACA CAG TCT ATG 0CC |
| 211 | ATC ATA CGT TAT ATA GCT GAC AAG CAC AAC ATG TTG GGT GGT |
| 253 | TGT CCA AAA GAG CGT GCA GAG ATT TCA ATG CTT GAA GGA GCG |
| 295 | GTT TTG GAT ATT AGA TAC GGT GTT TCG AGA ATT GCA TAT AGT |
| 337 | AAA GAC TTT GAA ACT CTC AAA GTT GAT TTT CTT AGC AAG CTA |
| 379 | CCT GAA ATG CTG AAA ATG TTC GAA GAT CGT TTA TGT CAT AAA |
| 421 | ACA TAT TTA AAT GGT GAT CAT GTA ACC CAT CCT GAC TTC ATG |
| 463 | TTG TAT GAC GCT CTT GAT GTT GTT TTA TAC ATG GAC CCA ATG |
| 505 | TGC CTG GAT GCG TTC CCA AAA TTA GTT TGT TTT AAA AAA CGT |
| 547 | ATT GAA GCT ATC CCA CAA ATT GAT AAG TAC TTG AAA TCC AGC |
| 589 | AAG TAT ATA GCA TGG CCT TTG CAG GOC TGG CAA GCC ACG TTT |
| 631 | GGT GGT GGC GAC CAT CCT CCA AAA TCG GAT CTG GAA GTT CTG |
| 673 | TTC CAG GGG CCC CTG GGA TCC CCA GGA ATT CCC 000 ATG GCA |
| 715 | AAC TCA ATG AAT GGC AGA AAC CCT GGT GOT CGA GGA GGA AAT |
| 757 | CCC CGA AAA GGT CGA ATT TTG GGT ATT ATT GAT GCT ATT CAG |
| 799 | GAT GCA GTT GGA CCC CCT AAG CAA GCT GCC GCA GAT CGC AGG |
| 841 | ACC GTG GAG AAG ACT TGG AAG CTC ATG GAC AAA GTG GTA AGA |
| 883 | CTG TGC CAA AAT CCC AAA CTT CAG TTG AAA AAT AGC CCA CCA |
| 925 | TAT ATA CTT GAT ATT TTG CCT GAT ACA TAT CAG CAT TTA CGA |
| 967 | CTT ATA TTG AGT AAA TAT GAT GAC AAC CAG AAA CTT GCC CAA |
| 1009 | CTC AGT GAG AAT GAG TAC TTT AAA ATC TAC ATT GAT AGC CTT |
| 1051 | ATG AAA AAG TCA AAA CGG GCA ATA AGA CTC TTT AAA GAA GGC |
| 1093 | AAG GAG AGA ATG TAT GAA GAA CAG TCA CAG GAC AGA CGA AAT |
| 1135 | CTC ACA AAA CTG TCC CTT ATC TTC AGT CAC ATG CTG GCA GAA |
| 1177 | ATC AAA GCA ATC TTT CCC AAT GGT CAA TTC CAG GGA GAT AAC |
| 1219 | TTT CGT ATC ACA AAA GCA GAT GCT GCT GAA TTC TGG AGA AAG |
| 1261 | TTT TTT GGA GAC AAA ACT ATC GTA CCA TGG AAA GTA TTC AGA |
| 1303 | CAG TGC CTT CAT GAG GTC CAC CAG ATT AGC TCT GGC CTG GAA |
| 1345 | GCA ATG GCT CTA AAA TCA ACA ATT GAT TTA ACT TGC AAT GAT |
| 1387 | TAC ATT TCA GTT TTT GAA TTT GAT ATT TTT ACC AGG CTG TTT |
| 1429 | CAG CCT TGG GGC TCT ATT TTG CGG AAT TGG AAT TTC TTA GCT |
| 1471 | GTG ACA CAT CCA GGT TAC ATG GCA TTT CTC ACA TAT GAT GAA |
| 1513 | GTT AAA GCA CGA CTA CAG AAA TAT AGC ACC AAA CCC GGA AGC |
| 1555 | TAT ATT TTC CGG TTA AGT TGC ACT CGA TTG GGA CAG TGG GCC |
| 1597 | ATT GGC TAT GTG ACT GGG GAT GGG AAT ATC TTA CAG ACC ATA |
| 1639 | CCT CAT AAC AAG CCC TTA TTT CAA GCC CTG ATT GAT GGC AGC |
| 1681 | AGG GAA GGA TTT TAT CTT TAT CCT GAT GGG AGG AGT TAT AAT |
| 1723 | CCT GAT TTA ACT GGA TTA TGT GAA CCT ACA CCT CAT GAC CAT |
| 1765 | ATA AAA GTT ACA CAG GAA CAA TAT GAA TTA TAT TGT GAA ATG |
| 1807 | GGC TCC ACT TTT CAG CTC TGT AAG ATT TGT GCA GAG AAT GAC |
| 1849 | AAA GAT GTC AAG ATT GAG CCT TGT GGG CAT TTG ATG TGC ACC |

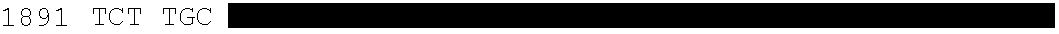

| | |
|---|---|
| 1933 | TGC CCT TTC TGT CGT TGT GAA ATA AAA GGA ACT GAG CCC ATA |
| 1975 | ATC GTG GAT CCC TTT GAT CCA AGA GAT GAA GGC TCC AGG TGT |
| 2017 | TGC AGC ATC ATT GAC CCC TTT GGC ATG CCG ATG CTC GAC TTG |
| 2059 | GAC GAC GAT GAT GAT CGT GAG GAG TCC TTG ATG ATG AAT CGG |
| 2101 | TTG GCA AAC GTC CGA AAG TGC ACT GAC AGG CAG AAC TCA CCA |
| 2143 | GTC ACA TCA CCA GGA TCC TCT CCC CTT GCC CAG AGA AGA CTC |
| 2185 | GAG CGG CCG CAT CGT GAC TGA |

Protein sequence (SEQ ID NO: 100):

| | |
|---|---|
| 1 | MSPILGYWKI KGLVQPTRLL LEYLEEKYEE HLYERDEGDK WRNKKFELGL |
| 51 | EFPNLPYYID GDVKLTQSMA IIRYIADKHN MLGGCPKERA EISMLEGAVL |
| 101 | DIRYGVSRIA YSKDFETLKV DFLSKLPEML KMFEDRLCHK TYLNGDHVTH |
| 151 | PDFMLYDALD VVLYMDPMCL DAFPKLVCFK KRIEAIPQID KYLKSSKYIA |
| 201 | WPLQGWQATF GGGDHPPKSD LEVLFQGPLG SPGIPGMANS MNGRNPGGRG |
| 251 | GNPRKGRILG IIDAIQDAVG PPKQAAADRR TVEKTWKLMD KVVRLCQNPK |
| 301 | LQLKNSPPYI LDILPDTYQH LRLILSKYDD NQKLAQLSEN EYFKIYIDSL |
| 351 | NKKSKPAIRL FKEGKERMYE EQSQDRRNLT KLSLIFSHML AEIKAIFPNG |
| 401 | QFQGDNFRIT KADAAEFWRK FFGDKTIVPW KVFRQCLHEV HQISSGLEAN |
| 451 | ALKSTIDLTC NDYISVFEFD IFTRLFQPWG SILRNWNFLA VTHPGYMAFL |
| 501 | TYDEVKARLQ KYSTKPGSYI FRLSCTRLGQ WAIGYVTGDG NILQTIPHNK |
| 551 | PLFQALIDGS REGFYLYPDG RSYNPDLTGL CEPTPHDHIK VTQEQYELYC |

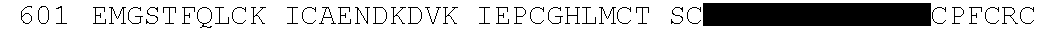

| | |
|---|---|
| 651 | EIKGTEPIIV DPFDPRDEGS RCCSIIDPFG MPMLDLDDDD DREESLMMNR |
| 701 | LANVRKCTDR QNSPVTSPGS SPLAQRRLER PHRD |

### EXAMPLE 3

In Vitro Self-Ubiquitination Assays

Self-ubiquitination was determined by homogenous time-resolved fluorescence resonance energy transfer assay (TR-FRET). The conjugation of ubiquitin cryptate to a specific GST-E3 and the binding of anti-GST tagged XL665 bring the two fluorophores into close proximity, which allows the FRET reaction to occur.

To measure hPOSH ubiquitination activity, GST-hPOSH or GST-hMDM2,GST-c-Cbl, GST-Cbl-b, GST-hPOSH2 and GST-POSH containing loop 3 of Cbl-b RING domain (10 uM) was incubated in reaction buffer (40 mM Hepes-NaOH, pH 7.5, 1 mM DTT, 2 mM ATP, 5 mM MgCl₂) with recombinant E1 (2 uM), UbcH5c (32 uM), ubiquitin (96 uM) and ubiquitin-cryptate (42 uM) (CIS bio International) for 30 minutes at 37° C. Reactions were stopped with 0.5M EDTA. Anti-GST-XL₆₆₅ (CIS bio International) (50 nM) was then added to the reaction mixture for further 45 minutes incubation at room temperature. Emission at 620 nm and 665 nm was obtained after excitation at 380 nm in a fluorescence reader (RUBYstar, BMG Labtechnologies). The generation of E3 ubiquitin-cryptate adducts was then determined by calculating the fluorescence resonance energy transfer (FRET=ΔF) using the following formula:
ΔF=[(S665/S620−B665/B620)/(C665/C620−B665/B620)]
where: S=actual fluorescence, B=Fluorescence obtained in parallel incubation without hPOSH, C=Fluorescence obtained in reaction without added compounds.

Identical assays were performed to measure GST-hMdm2 and GST-c-Cbl ubiquitination activities.

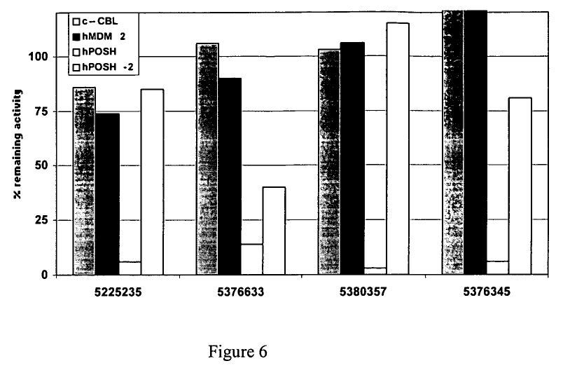

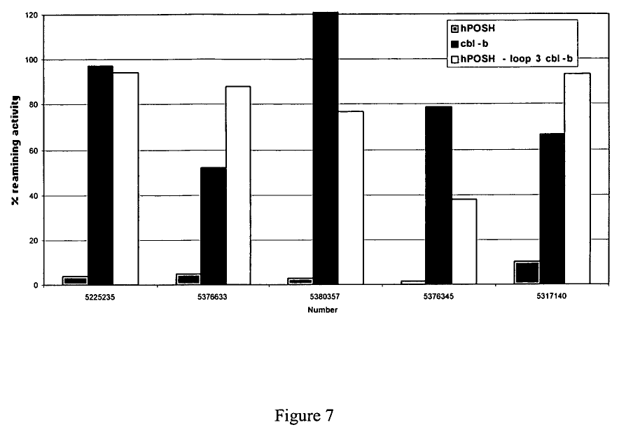

Assays as described above were conducted in the absence and presence of the commercially available compounds depicted in Table 2 below. Results are presented in FIGS. 6 and 7.

TABLE 2

| Compound ID | CAS number | Structure | MW | ref |
|---|---|---|---|---|
| 5317140 | 38307-83-4 | 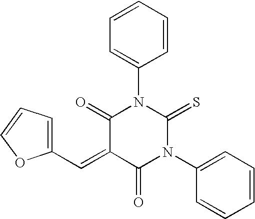 | 374 | Ger. Offen. (1972) DE 2042663 Photographic dry |
| 5376633 | 356792-81-9 | 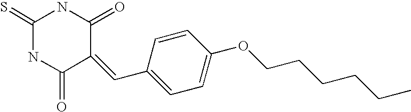 | 332 | No ref |
| 5376345 | 3412944-98-2 | 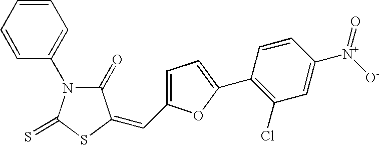 | 442 | No ref |
| 5380357 | 525569-45-3 | 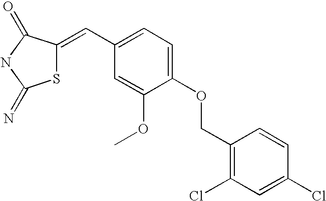 | 409 | No ref |
| 5225235 | 413595-33-2 | 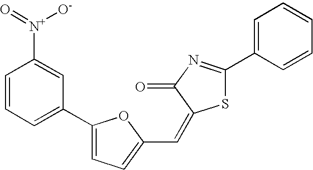 | 376 | No ref |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A RING finger E3 ligase selective inhibitor, wherein the RING finger E3 ligase selective inhibitor interacts with a specificity domain of a RING finger E3 ligase.

2. A method of identifying the specificity domain of a RING Finger E3 ligase comprising:
(a) identifying the RING domain of the RING finger E3 ligase; and
(b) determining the position of the conserved Cysteine residues,
wherein the specificity domain is the domain that lies between the 6^{th} and 7^{th} conserved Cysteine residue.

3. An isolated or recombinant polypeptide selected from the group consisting of:
(a) a polypeptide consisting essentially of a specificity domain;
(b) a fusion protein comprising a specificity domain of a RING finger E3 ligase and a second domain; and
(c) a chimeric RING Finger E3 ligase in which the specificity domain is replaced by the specificity domain of a second RING Finger E3 ligase.

4. The polypeptide of claim 3, wherein the specificity domain is selected from the group consisting of: SEQ ID Nos. 1-84.

5. A method of inhibiting interaction of a RING Finger E3 ligase and a RING Finger E3 ligase associated protein, comprising administering to a subject in need thereof an agent that interacts with the specificity domain of the RING finger E3 ligase.

6. A method of identifying an inhibitor of a RING finger E3 ligase, comprising:
(a) identifying the specificity domain of the RING finger E3 ligase; and
(b) identifying an agent that binds to at least a portion of the specificity domain, wherein the agent identified in (b) is an inhibitor of the RING finger E3 ligase.

7. The method of claim 6, wherein the inhibitor of the RING finger E3 ligase is a selective inhibitor with respect to other RING finger E3 ligases.

8. The method of claim 6, wherein the inhibitor of the RING finger E3 ligase is a small molecule comprising a selective binding element and a functional inhibitory element.

9. The method of claim 6, wherein only the selective binding element binds to the specificity domain of the RING finger E3 ligase.

10. The method of claim 6, wherein the inhibitor selectively inhibits the interaction of the RING finger E3 ligase with a RING finger E3 ligase associated protein.

11. The method of claim 6, wherein the specificity domain is selected from the group consisting of: SEQ ID Nos. 1-84.

12. The method of claim 6, wherein (b) comprises:
(i) forming a mixture comprising: a polypeptide comprising a portion of at least 20 amino acids of a RING finger E3 ligase, wherein the polypeptide includes at least a portion of the specificity domain of the RING finger E3 ligase; an E2; and a test agent; and
(ii) detecting one or more of the following: binding of the test agent to the polypeptide, or binding of E2 to the polypeptide.

13. The method of claim 6, which method further comprises:
(c) forming a mixture comprising the RING finger E3 ligase, or the RING domain thereof; an E1; an E2; ubiquitin; and a test agent; and
(d) detecting the effect of the test agent on ubiquitination of the RING finger E3 ligase.

14. The method of claim 6, which method further comprises:
(c) forming a mixture comprising the RING finger E3 ligase, or the RING domain thereof; an E1; an E2; ubiquitin; a substrate of the RING finger E3 ligase; and a test agent; and
(d) detecting the effect of the test agent on ubiquitination of the substrate.

15. The method of claim 6, wherein the inhibitor selectively inhibits the interaction of the RING finger E3 ligase with an E2.

16. The method of claim 6, wherein the inhibitor selectively inhibits the interaction of the RING finger E3 ligase with a first E2 verus a second E2.

17. The method of claim 6, wherein the inhibitor selectively inhibits the ubiquitin ligase activity of the RING finger E3 ligase.

18. The method of claim 6, wherein the inhibitor selectively inhibits the ubiquitin ligase activity of the RING finger E3 ligase versus the ubiquitin ligase activity of another RING finger E3 ligase.

19. The method of claim 6, wherein (b) is conducted by high throughput screening.
